(19) United States
(12) Patent Application Publication
(10) Pub. No.: US 2009/0226418 A1
(43) Pub. Date: Sep. 10, 2009

(54) Food Products Comprising Probiotic Micro-Organisms and Antibodies

(76) Inventors: Leo Gerardus Frenken, Vlaardingen (NL); Lars-Göran Lennart Hammarström, Huddinge (SE); Adrianus Marinus Ledeboer, Vlaardingen (NL)

Correspondence Address: MORGAN LEWIS & BOCKIUS LLP, 1111 PENNSYLVANIA AVENUE NW, WASHINGTON, DC, 20004, US

(21) Appl. No.: 11/990,214

(22) PCT Filed: May 24, 2006
(86) PCT No.: PCT/EP2006/005060

§ 371 Date: Feb. 8, 2008

(30) Foreign Application Priority Data
Aug. 19, 2005 (EP) 05076940.5

(51) Int. Cl. A61K 39/395; A61K 35/74; A23J 1/00; A23L 1/00; A23L 1/28; A23K 1/18

(52) U.S. Cl. 424/130.1; 424/93.2; 424/93.21; 426/656; 426/89; 426/61; 426/62; 426/2

(57) ABSTRACT

The present invention relates to food products or pharmaceutical preparations comprising antibodies or antibody fragments which are active in the gut and probiotic micro-organisms independent from the antibodies or antibody fragments. In particular, the invention relates to a method for preparing food products and pharmaceutical preparations comprising the antibodies or antibody fragments and probiotic micro-organisms and the use of these products to deliver health benefits to humans.

### FIELD OF THE INVENTION

The present invention relates to food products or pharmaceutical preparations comprising antibodies or antibody fragments which are active in the gut and probiotic micro-organisms independent from the antibodies or antibody fragments. In particular, the invention relates to a method for preparing food products and pharmaceutical preparations comprising the antibodies or antibody fragments and probiotic micro-organisms and the use of these products to deliver health benefits to humans.

### BACKGROUND OF THE INVENTION

Antibodies (also called immunoglobulins) are glycoproteins, which specifically recognise foreign molecules. These recognised foreign molecules are called antigens. When antigens invade humans or animals, an immunological response is triggered which involves the production of antibodies by B-lymphocytes. By this immunological response, microorganisms, larger parasites, viruses and bacterial toxins can be rendered harmless. The unique ability of antibodies to specifically recognise and bind with high affinity to virtually any type of antigen, makes them useful molecules in medical and scientific research.

In vertebrates five immunoglobulin classes are described, including IgG, IgM, IgA, IgD and IgE, all of which differ in their function in the immune system. IgGs are the most abundant immunoglobulins in the blood. They have a basic structure of two identical heavy (H) chain polypeptides and two identical light (L) chain polypeptides. The H and L chains are kept together by disulfide bridges and non-covalent bonds. The chains themselves can be divided in variable and constant domains. The variable domains of the heavy and light chain (V_{H} and V_{L}) which are extremely variable in amino acid sequences are located at the N-terminal part of the antibody molecule. V_{H} and V_{L} together form the unique antigen-recognition site. The amino acid sequences of the remaining C-terminal domains are much less variable and are called C_{H}1, C_{H}2, C_{H}3 and C_{L}.

The non-antigen binding part of an antibody molecule is called the constant domain Fc and mediates several immunological functions, such as binding to receptors on target cells and complement fixation.

The unique antigen-binding site of an antibody consists of the heavy and light chain variable domains (V_{H} and V_{L}). Each domain contains four framework regions (FR) and three regions called CDRs (complementarity determining regions) or hypervariable regions. The CDRs strongly vary in sequence and determine the specificity of the antibody. V_{L} and V_{H} domains together form a binding site, which binds a specific antigen.

Several functional antigen-binding antibody fragments could be engineered by proteolysis of antibodies, using for example papain digestion, pepsin digestions or other enzymatic approaches. Such a technique can be used to yield Fab, Fv or single domain fragments.

Fab fragments are the antigen-binding domains of an antibody molecule. Fab fragments can be prepared by papain digestions of whole antibodies. Fv fragments are the minimal fragment (˜30 kDa) that still contains the whole antigen-binding site of a whole IgG antibody. Fv fragments are composed of both the variable heavy chain (V_{H}) and variable light chain (V_{L}) domains. This heterodimer, called Fv fragment (for fragment variable) is still capable of binding the antigen.

Hence, smaller antibody fragments may be synthesised and these fragments will have an advantage over whole antibodies in applications requiring tissue penetration and rapid clearance from the blood or kidney.

The in vitro production of antibodies has been possible since the development of monoclonal antibody technology. This has led to the use of antibodies in many areas including research, medicine and recently in consumer applications. However, such applications rely of the large scale production of antibodies and generally involve the use of the antibody or antibody fragment per se i.e the harvested protein from an antibody expression system.

Escherichia coli has been used as an expression system for antibody fragment production. E. coli is easily accessible for genetic modifications, requires simple inexpensive media for rapid growth and they can easily be cultured in fermentors permitting large-scale production of proteins of interest.

Furthermore, in a recent article, “in situ delivery of passive immunity by lactobacilli producing single-chain antibodies” Nature Biotechnol. (2002) 20, 702-706, Kruger et al reported on the production of scFv antibody fragments against Streptococcus mutans by the Gram positive food grade bacteria Lactobacillus zeae. This treatment involved in situ delivery of passive immunity at oral mucosal sites only wherein the single chain antibody fragments were shown to deliver protection against dental caries in rats.

Lactobacilli have been investigated with regards to their anti-diarrhoeal properties since the 1960's (Beck, C., et al. Beneficial effects of administration of Lactobacillus acidophilus in diarrhoeal and other intestinal disorders. Am. J. Gastroenterol (1961) 35, 522-30). A limited number of recent controlled trials have shown that certain strains of lactobacilli may have therapeutic as well as prophylactic properties in acute viral gastroenteritis (Mastretta, E., et al. Effect of Lactobacillus CG and breast-feeding in the prevention of rotavirus nosocomial infection. J. Pediatr. Gastroenterol. Nutr. (2002) 35, 527-531). Selected strains of Lactobacillus casei and Lactobacillus plantarum have also been shown to exert strong adjuvant effects on the mucosal and the systemic immune response.

Lactobacilli are well-known bacteria applied in the production of food products. For example yoghurt is normally made by fermenting milk with among others a Lactobacillus strain. The fermented acidified product, still containing the viable Lactobacillus, is then cooled and consumed at the desired moment.

Another application of Lactobacillus in food products is in the production of meat products for example sausages. Here the Lactobacillus is added to the meat mass prior to applying the casing, followed by a period of ripening in which the fermentation process takes place.

Still another application of Lactobacillus in the production of food products is the brining of vegetables such as cabbage (sauerkraut), carrots, olives or beets. Here the natural fermentation process can be controlled by the addition of an appropriate Lactobacillus starter culture.

The application of Lactobacillus in food products is often associated with several health effects, see for example A. C. Ouwehand et al. in Int. Dairy Journal 8 (1998) 749-758. In particular the application of products is associated with several health effects for example relating to gut well being such as IBS (Irritable Bowel Syndrome), reduction of lactose maldigestion, clinical symptoms of diarrhoea, immune stimulation, anti-tumour activity and enhancement of mineral uptake.

WO 99/23221 discloses multivalent antigen binding proteins for inactivating phages. The hosts may be lactic acid bacteria which are used to produce antibody binding fragments which are recovered. WO 99/23221 discloses adding the harvested antibody fragments to bacteria to provide anti-diarrhoea effects.

WO 00/65057 is directed to the inhibition of viral infection, using monovalent antigen-binding proteins. The antigen-binding protein may be a heavy chain variable domain derived from an immunoglobulin naturally devoid of light chains, such as those derived from Camelids as described in WO 94/04678. WO 00/65057 discloses transforming a host with a gene encoding the monovalent antigen-binding proteins. Suitable hosts can include lactic acid bacteria. This disclosure relates to the field of fermentation processing and the problem of phage infection which hampers fermentation. Specifically, llama VHH fragments are used to solve the problem of phage infection by neutralising Lactoccoccus lactis bacteriophage P2.

Both WO 00/65057 and WO 99/23221 involve the use of antibody fragments harvested from a bacterial expression system.

U.S. Pat. No. 6,605,286 is directed to the use of gram positive bacteria to deliver biologically active polypeptides, such as cytokines, to the body. U.S. Pat. No. 6,190,662 and EP 0 848 756 B1 are directed to methods for obtaining surface expression of a desired protein or polypeptide. Monedero et al “Selection of single-chain antibodies against the VP8 Subunit of rotavirus VP4 outer capsid protein and their expression in L. casei” Applied and Environmental Microbiology (2004) No. 4 6936-6939, is directed to in-vitro studies on the use of single-chain antibodies (scFv) expressed by L. casei which recognise the VP8 and fraction of rotavirus outer capsid and blocks rotavirus infection in vitro. However, none of these documents disclose the use of heavy chain immunoglobulins or fragments of the VHH or VNAR type, or domain antibodies (dAbs) of the heavy or light chains of immunoglobulins or fragments thereof.

Other micro-organisms used in the production of food products include yeasts. Yeast is well-known in the brewing and baking and their associated food products including, for example bread and beer.

One disadvantage of these known systems is that the use of antibodies or antibody fragments per se (i.e. the harvested protein) in the treatment of a disease in a human may result in the antibody being degraded or digested before they provide the desired health benefits and even before they reach the desired location.

Furthermore, it is often desirable to ensure that the antibody or antibody fragments are active in a specific region of the body, for example the gut.

Additionally, it is desirable for the health benefits to be provided to be as beneficial as possible.

Hence, the present invention aims to provide health benefits to a subject in need thereof.

### SUMMARY OF THE INVENTION

According to a first aspect of the invention, there is provided a food product or pharmaceutical preparation comprising i) antibodies or antibody fragments which are active in the gut and ii) probiotic micro-organisms independent from the antibodies or antibody fragments.

According to a second aspect of the invention, there is provided a method for making a food product or pharmaceutical preparation according to the first aspect comprising adding independently the antibodies or antibody fragments and the probiotic micro-organisms during the manufacture of the food product or pharmaceutical preparation or an ingredient thereof.

According to a third aspect of the invention, there is provided the use of the food product or pharmaceutical preparation according to the first aspect of the invention or made according to the second aspect of the invention to deliver health benefits to the gut of a subject.

According to a fourth aspect of the invention, there is provided a method of delivering health benefits to the gut of a subject comprising administering the food product or pharmaceutical preparation according to the first aspect of the invention or made according to the second aspect of the invention to a subject in need thereof.

According to a fifth aspect of the invention, there is provided a dispensing implement for use with a food product comprising probiotic micro-organisms wherein the dispensing implement is coated on at least one surface with antibodies or anti-body fragments which are active in the gut.

According to a sixth aspect of the invention, there is provided a dispensing implement for use with a food product wherein the dispensing implement is coated on at least one surface with antibodies or anti-body fragments which are active in the gut and probiotic micro-organisms.

For the avoidance of doubt, the term “food product” as used herein encompasses beverages.

By the term “non-viable bacteria” as used herein is meant a population of bacteria that is not capable of replicating under any known conditions. However, it is to be understood that due to normal biological variations in a population, a small percentage of the population (i.e. 5% or less) may still be viable and thus capable of replication under suitable growing conditions in a population which is otherwise defined as non-viable.

By the term “viable bacteria” as used herein is meant a population of bacteria that is capable of replicating under suitable conditions under which replication is possible. However, it is to be understood that due to normal biological variations in a population, a small percentage of the population (i.e. 5% or less) may still be non-viable and thus not capable of replication under those conditions in a population which is otherwise defined as viable.

By the term “probiotic micro-organisms independent from the antibodies or antibody fragments” as used herein is meant that the probiotic micro-organisms do not form a part of any delivery system for the antibodies or antibody fragments and are not binded therewith.

### BRIEF DESCRIPTION OF THE FIGURES

In the detailed description of the embodiments of the invention, reference is made to the following figures.

FIGS. 1a and b shows that rotavirus specific VHH particles neutralise rotavirus in-vitro.

FIG. 2 shows that rotavirus specific VHH particles neutralise rotavirus in-vivo.

FIG. 3 shows a Map of Lactobacillus expression vectors:

(a) 2A10-anchor;
(b) VHH1-anchor, mediating surface-anchored expression of antibody fragments by fusion to the last 244 amino acids of L. casei proteinase P;
(c) 2A10-secreted; and
(d) VHH1-secreted with a stop codon (TAA) inserted after the E-tag sequence, mediating secretion of the antibody fragment.
Tldh: transcription terminator of the lactate dehydrogenase gene of L. casei;
deleted Tld., remaining sequence after deletion of Tldh;
2A10-scFv: single-chain antibody against VP4/VP7;
VHH1: heavy chain antibody fragment against rotavirus; long anchor, anchor sequence from the proteinase P gene of L. casei (244 amino acids);
Tcbh: transcription terminator sequence of the conjugated bile acid hydrolase gene of L. plantarum 80;
Pldh: Promotor sequence of the lactate dehydrogenase gene of L. casei, SS
PrtP;
   signal sequence of the PrtP gene (33 aa), N-terminus PrtP, N terminus (36 amino cids) of the PrtP gene;
Amp^{r}: ampicillin-resistance gene;
Ery: erythromycin-resistance gene;
Rep: repA gene of plasmid p353-2 from L. pentosis;
Ori: origin of replication (Ori+=ori E. coli, Ori-=ori Lactobacillus).
   Arrows indicate a stop codon.

FIG. 4a shows the results of Flow cytometry showing the expression of 2A10-scFv (light grey) and VHH1 (dark grey) on the surface of the Lactobacillus paracasei by

[symbol]

FIG. 12 shows an alignment of the VHH's having affinity for Rotavirus viral particles.

### DETAILED DESCRIPTION OF THE INVENTION

The invention will now be described by way of example, by a series of embodiments.

In general, the present invention is directed to a food product or pharmaceutical preparation comprising i) antibodies or antibody fragments which are active in the gut and ii) probiotic micro-organisms independent from the antibodies or antibody fragments.

i) Antibodies or Antibody Fragments which are Active in the Gut

The antibodies or antibody fragments which are active in the gut may be used either as part of a delivery system therefor or not.

According to one embodiment of the present invention it is preferred that the antibodies or fragments thereof comprise part of a delivery system to deliver them to the GIT (hereinafter referred to as the “delivery system”).

According to aspect of this embodiment when using a delivery system for the antibodies or fragments thereof to deliver them to the GIT, this can be effected by the use of encapsulates, such as those known in the food and pharmaceutical industries. Natural biopolymers may be used. Examples include Ca-alginate, carrageenan, gellan gum or gelatine. The delivery system may be an encapsulation method known in the art which will deliver the immunoglobulin or fragments thereof specifically to the gut. The encapsulate must therefore be able to survive until entry to the gut and then be released. Such a delivery system comprises a general protective system that protects the antibodies from degradation. Such techniques may include liposome entrapment, spinning disk and coacervation. Any trigger can be used to prompt the release of the encapsulated ingredient, such as pH change (enteric coating), mechanical stress, temperature, enzymatic activity. These techniques are expanded on in the article by Sébastien Gouin “Microencapsulation: industrial appraisal of existing technologies and trends” Food Science and Technology (2004) 15: 330-347. Preferably, an enteric coating is used. Additionally, the encapsulation method may allow the slow release of the antibody in the gut and/or stomach. This will enable a constant release of the antibody or functional fragment or equivalent over a set period of time.

Alternatively, according to another aspect of this embodiment the delivery system may comprise a micro-organism, preferably transformed to be able to produce the antibodies or antibody fragments. This micro-organism is additional to the probiotic micro-organisms referred to herein as ii) and which is independent from the antibodies or antibody fragments. Thus for the avoidance of doubt, the invention may comprise two different micro-organisms. The first is the probiotic micro-organism referred to herein as ii) which does not form part of any delivery system for the antibodies or fragments thereof. The second is the micro-organism which may form part of the delivery system. The former is referred to herein as the “probiotic micro-organism” and the latter as the “micro-organism”.

According to a particular aspect of the present invention there is provided a pharmaceutical preparation comprising a delivery system for delivering antibodies to the GIT wherein the antibodies are active in the gut and the delivery system comprises a micro-organism transformed with antibodies or fragments thereof wherein the antibodies are heavy chain immunoglobulins of the VHH type or fragments thereof, preferably derived from Camelids, most preferably llama heavy chain antibodies or fragments thereof, or domain antibodies (dAbs) of the heavy or light chains of immunoglobulins or fragments thereof and independently a probiotic micro-organism.

Like the probiotic micro-organism, the micro-organism should preferably be able to survive passage in the GIT and should be active in the stomach/gut. Preferably, the micro-organism should be able to undergo transient colonization of the GIT; be able to express the gene in the GIT; and be able to stimulate the gut immune system.

Preferably, the micro-organism may also be a probiotic microorganism with the above characteristics. In this case there will be two probiotic micro-organisms used according to the invention; one which is independent of the antibodies or fragments thereof and one which forms part of a delivery system therefor. Probiotics are defined as viable microbial food supplements which beneficially influence the host by improving its intestinal microbial balance in accordance to Fuller (1989) probiotics in man and animals, Journal of Applied Bacteriology 66, 365-378. If the probiotic micro organism is a bacterium, it is preferred that it is a lactic acid bacterium.

Examples of other suitable probiotic micro-organisms include yeast such as Saccharomyces, Debaromyces, Kluyveromyces and Pichia, moulds such as Aspergillus, Rhizopus, Mucor and Penicillium and bacteria such as the genera Bifidobacterium, Propionibacterium, Streptococcus, Enterococcus, Lactococcus, Bacillus, Pediococcus, Micrococcus, Leuconostoc, Weissella, Oenococcus and Lactobacillus. Kluyveromyces lactis may also be used.

Specific examples of suitable probiotic microorganisms are:

Kluyveromyces lactis, Kluyveromyces fragilis, Pichia pastoris, Saccharomyces cerevisiae, Saccharomyces boulardii, Aspergillus niger, Aspergillus oryzae, Mucor miehei, Bacillus subtilis, Bacillus natto, Bifidobacterium adolescentis, B. animalis, B. breve, B. bifidum, B. infantis, B. lactis, B. Iongum, Enterococcus faecium, Enterococcus faecalis, Escherichia coli, Lactobacillus acidophilus, L. brevis, L. casei, L. delbrueckii, L. fermentum, L. gasseri, L. helveticus, L. johnsonii, L. lactis, L. paracasei, L. plantarum, L. reuteri, L. rhamnosus, L. sakei, L. salivarius, L. sanfranciscus, Lactococcus lactis, Lactococcus cremoris, Leuconostoc mesenteroides, Leuconostoc lactis, Pediococcus acidilactici, P. cerevisiae, P. pentosaceus, Propionibacterium freudenreichii, Propionibacterium shermanii and Streptococcus salivarius.

Particular probiotic strains are:

Saccharomyces boulardii, Lactobacillus casei shirota, Lactobacillus casei immunitas, Lactobacillus casei DN-114 001, Lactobacillus rhamnosus GG (ATCC53103), Lactobacillus reuteri ATCC55730/SD2112, Lactobacillus rhamnosus HN001, Lactobacillus plantarum 299v (DSM9843), Lactobacillus johnsonii La1 (I-1225 CNCM), Lactobacillus plantarum WCFS1, Bifidobacterium lactis HN019, Bifidobacterium animalis DN-173010, Bifidobacterium animalis Bb12, Lactobacillus casei 431, lactobacillus acidophilus NCFM, Lactobacillus reuteri ING1, Lactobacillus salivarius UCC118, Propionibacterium freudenreichi JS, Escherichia coli Nissle 1917.

Conveniently, the micro-organism may be a lactic acid bacterium. More, preferably, the micro-organism is chosen from either lactobacillus or bifidobacteria. Even more preferably, the micro-organism is Lactobacillus. Particularly, the Lactobacillus is Lactobacillus casei 393 pLZ15. Lactobacillus casei has recently been reidentified as Lactobacillus paracasei (Perez-Martinez, 2003). Another preferred Lactobacillus is Lactobacillus reutarii.

Alternatively, the micro-organism may be yeast. Suitable yeasts include the bakers yeast S. cerevisiae. Other yeasts like Candida boidinii, Hansenula polymorpha, Pichia methanolica and Pichia pastoris which are well known systems for the production of heterologous proteins and may be used in the present invention.

Filamentous fungi, in particular species from the genera Trichoderma and Aspergillus have the capacity to secrete large amounts of proteins, metabolites and organic acids into their culture medium. This property has been widely exploited by the food and beverage industries where compounds secreted by these filamentous fungal species have been used for decades.

A delivery system based on probiotic bacteria represents a safe and attractive approach and represents one of the cheapest antibodies production systems. The wide scale application of the micro-organism, preferably Lactobacillus, expressing antibodies is relatively easy and requires minimal handling and storage costs and economical.

Preferably, the micro-organism is transformed with an expression vector comprising the gene for the antibody. The expression vector may contain a constitutive promoter in order to express the antibodies or fragments thereof. Such a constitutive promoter will support in situ expression of antibodies by transformed lactobacilli persisting (at least for a short period) in the intestinal tract after administration. Alternatively, the promoter may be chosen to be active only in the GIT and/or stomach/gut i.e. suitable for GIT specific expression only. This will ensure expression and/or secretion of the llama heavy chain antibody or fragments thereof in the GIT, preferably the gut. Many constitutive promoters for lactobacilli are known in the art and an example of a promoter that is specifically inducible in the GIT is Pldh (Pouwels et al “Lactobacilli as vehicles for targeting antigens to mucosal tissues by surface exposition of foreign antigens” Methods in Enzymology (2001) 336:369-389).

The expression vectors described in the examples are able to replicate in the transformed lactobacilli and express the antibodies of fragments thereof. It will be understood that the present invention is not limited to these replication expression vectors only. The whole expression cassette can be inserted in a so-called “integration” plasmid, whereby the expression cassette will be integrated into the chromosome of the lactobacilli after transformation, as known in the art (Pouwels, P. H. and Chaillou, S. Gene expression in lactobacilli (2003) Genetics of lactic acid bacteria page 143-188). Thus, replicating or integrating vectors may be used in accordance with the invention.

When the delivery system comprises a micro-organism transformed with antibodies or fragments thereof the antibodies are expressed and/or secreted in the gut. Hence, use of a micro-organism as the delivery system has the advantages that in vivo production of antibody fragments locally in the GIT circumvents the practical problem of degradation of orally administered antibodies in the stomach. Such a system based on probiotic bacteria represents a safe and attractive approach to delivering antibodies to the GIT. Hence, the wide scale application of the lactobacilli expressing antibodies is relatively easy and requires minimal handling and storage costs and economical. Furthermore, the probiotic bacteria will remain in the gut for longer and enable the constant production of the antibody to enable more constant protection against the enteropathogenic microorganism.

Advantageously the amount of the micro-organism in the delivery system in food products of the invention is between 10⁶ and 10¹¹ per serving or (for example if serving size is not known) between 10⁶ and 10¹¹ per 100 g of product, more preferred these levels are from 10⁸ to 10⁹ per serving or per 100 g of product.

The antibodies for use according to the present invention must be active in the gut/stomach, i.e. they must be functional and retain their normal activity including inactivating their target. The active antibodies according to the invention should bind to their target as normal, thus, the binding affinity of the antibody for the antigen should be as normal. Binding affinity is present when the dissociation constant is more than 10⁵.

Hence, the food product or pharmaceutical preparation according to the invention will be able to selectively address a specific disease or symptom of a disease. The choice of antibody will determine the disease or symptom to be treated or reduced.

It will be understood that when the product is a food product any antibody may be used. However, when the product is a pharmaceutical preparation heavy chain immunoglobulins or fragments thereof of the VHH or VNAR type or domain antibodies (dAbs) of the heavy or light chains of immunoglobulins or fragments thereof are preferred.

Preferably, the antibody or fragments thereof should have one or more of the following characteristics:
i). They show good binding affinity and the desired inhibition functionality under the conditions present in the G/I tract; and
ii) They have good proteolytic stability in that they are stable against degradation by proteolytic enzymes.
iii) The antibodies should be thermostable which enables their inclusion in a variety of food products. The food products may be prepared in a process requiring pasteurization and it is preferred that the activity of the antibodies is largely maintained despite heat treatment.

The use of fragments or portions of a whole antibody which can nevertheless exhibit antigen binding affinity is also contemplated. Fragments should preferably be functional fragments. A functional fragment of an immunoglobulin means a fragment of an immunoglobulin which fragment show binding affinity for an antigen and has the same biological activity as the full length sequence. Such fragments include Fab and scFv fragments. Binding affinity is present when the dissociation constant is more than 10exp5. Such a fragment can be advantageously used in therapy, for example, as it is likely to be less immunogenic and more able to penetrate tissues due to its smaller size.

Functional equivalents are also contemplated. A functional equivalent means a sequence which shows binding affinity for an antigen similar to the full length sequence. For example, additions or deletions of amino acids which do not result in a change of functionality are encompassed by the term functional equivalents.

The antibody or fragment thereof should be able to be expressed and secreted in the gut. Several assays are well known in the art which mimic GIT conditions and are used for instance to select suitable probiotics that can survive GIT conditions. A suitable assay for determining whether an antibody can survive the GIT conditions is described by Picot, A. and Lacroix, C. (International Dairy Journal 14 (2004) 505-515).

In order to determine whether an antibody will be suitable for use in the present invention the following test may be applied. The antibody produced is selected under specific conditions of low pH, preferably from 1.5 to 3.5, and in the presence of pepsin (a protease abundant in the stomach) to result in highly beneficial molecules that work well in the G/I tract and are suitable for use according to the present invention.

The antibody or fragment thereof may be naturally occurring or may be obtained by genetic engineering using techniques well known in the art.

The antibody can be chosen to be active against many different antigens, including micro-organisms, larger parasites, viruses and bacterial toxins.

The present application may be applicable to the management of enteropathogenic micro-organisms in general. Enteropathogenic micro-organisms include viruses or enteropathogenic bacteria. Management is understood to mean therapy and/or prophylaxis.

Enteropathogenic bacteria may include, for example, Salmonella, Campilobacter, E. coli or Helicobacter. The use of antibodies that inactivate Helicobacter that causes stomach ulcers is contemplated.

Enteropathogenic viruses may include, for example, Norovirus (Norwalk like virus), enteric adenovirus, Coronavirus, astroviruses, caliciviruses, and parvovirus. Rotavirus and the Norwalk family of viruses are the leading causes of viral gastroenteritis, however, a number of other viruses have been implicated in outbreaks. Most preferably, the present invention is directed to the management of rotaviral infection.

The present application may also be used in the management of other non-enteropathogenic viruses like Hepatitis.

Preferably, heavy chain immunoglobulins or fragments thereof of the VHH or VNAR type or domain antibodies of the heavy or light chains of immunoglobulins or fragments thereof may be used in the present invention. Such heavy chain immunoglobulins of the VHH or VNAR type are obtained using techniques well known in the art. More preferably, the immunoglobulin or fragment thereof is derived from Camelids, most preferably llamas.

Van der Linden, R. H., et al. Comparison of physical properties of llama VHH antibody fragments and mouse monoclonal antibodies Biochim, Biophys. Acta (1990) 1431, 3746 obtained heavy chain antibodies with a high specificity and affinity against a variety of antigens. Furthermore, heavy chain immunoglobulins are readily cloned and expressed in bacteria and yeast as shown in Frenken, L. G. J., et al. “Isolation of antigen specific Llama V_{HH} antibody fragments and their high level secretion by Saccharomyces cerevisiae”. J. Biotechnol. (2000) 78, 11-21. Methods for the preparation of such immunoglobulins or fragments thereof on a large scale comprising transforming a mould or a yeast with an expressible DNA sequence encoding the antibody or fragment are also described in patent application WO 94/25591 (Unilever). Finally, EP-A-0584421 describes heavy chain immunoglobulin regions obtained from Camelids.

Preferably, the antibodies may be llama heavy chain antibodies, more preferably VHH antibodies or fragments thereof. In 1993, Hamers-Casterman et al. discovered a novel class of IgG antibodies in Camelidae i.e. camels, dromedaries and llamas. (“Naturally occurring antibodies of devoid light-chains” Nature (1993) 363, 446-448). Heavy chain antibodies constitute about one fourth of the IgG antibodies produced by the camelids, llamas. These antibodies are formed by two heavy chains but are devoid of light chains. The variable antigen binding part is referred to as the VHH domain and it represents the smallest naturally occurring, intact, antigen-binding site (Desmyter, A., et al. “Antigen specificity and high affinity binding provided by one single loop of a camel single-domain antibody” J. Biol. Chem. (2001) 276, 26285-26290). Heavy chain antibodies with a high specificity and affinity can be generated against a variety of antigens and they are readily cloned and expressed in bacteria and yeast (Frenken, L. G. J., et al. “Isolation of antigen specific Llama V_{HH} antibody fragments and their high level secretion by Saccharomyces cerevisiae” J. Biotechnol. (2000) 78, 11-21). Their levels of expression, solubility and stability are significantly higher than those of classical F(ab) or Fv fragments (Ghahroudi, M. A. et al “Selection and identification of single domain antibody fragments from camel heavy-chain antibodies” FEBS Lett. (1997) 414, 521-526).

Another good source of heavy chain antibodies can be found in sharks. It recently has been shown that sharks also have a single VH-like domain in their antibodies termed VNAR (Nuttall et al. “Isolation and characterization of an IgNAR variable domain specific for the human mitochondrial translocase receptor Tom70” Eur. J. Biochem. (2003) 270, 3543-3554; Dooley et al. “Selection and characterization of naturally occurring single-domain (IgNAR) antibody fragments from immunized sharks by phage display” Molecular Immunology (2003) 40, 25-33; Nuttall et al. “Selection and affinity maturation of IgNAR variable domains targeting Plasmodium falciparum AMA1” Proteins: Structure, Function and Bioinformatics (2004) 55, 187-197). Fragments of the VNAR-type-immunoglobulin can be used.

Holt et al, “Domain antibodies:proteins for therapy” Trends in Biotechnology (2003):Vol. 21, No. 11:484-490, reviews antigen-binding fragments called “domain antibodies” or dAbs which comprise only the V_{H} or V_{L} domain of an antibody and are consequently smaller than, for example, Fab and scFv. DAbs are the smallest known antigen-binding fragments of antibodies, ranging from 11 kDa to 15 kDa. They are highly expressed in microbial cell culture. Each dAb contains three of the six naturally occurring complementarity determining regions (CDRs) from an antibody.

The immunoglobulin or fragment thereof may be monovalent, multivalent (multispecific), i.e. bivalent, trivalent, tetravalent, in that it comprises more than one antigen binding site. The antigen binding sites may be derived from the same parent antibody or fragment thereof or from different antibodies which bind the same epitope. If all binding sites have the same specificity then a monospecific immunoglobulin is produced. Alternatively a multispecific immunoglobulin may be produced binding to different epitopes of the same antigen or even different antigens. It is preferred that the or at least one of the, binding sites is directed to pathogens (or products thereof such as enzymes produced therefrom) found in the gastro-intestinal tract. If is further preferred that the immunoglobulin or fragment thereof binds to rotavirus and more preferably that it neutralises it.

The immunoglobulin or fragment thereof of the VHH- or VNAR-type, or domain antibodies (dAbs) of the heavy or light chains of immunoglobulins or fragments thereof, may be naturally occurring i.e. elicited in vivo upon immunizing an animal with the desired antigen or synthetically made, i.e. obtained by genetic engineering techniques.

Techniques for synthesising genes, incorporating them into micro-organism hosts and expressing genes in micro-organisms are well known in the art and the skilled person would readily be able to put the invention into effect using common general knowledge. The use of replicating or integrating vectors is contemplated.

According to one embodiment of the present invention, the food product or pharmaceutical preparation comprises antibodies which are heavy chain immunoglobulins or fragments thereof of the VHH- or VNAR-type, or domain antibodies (dAbs) of the heavy or light chains of immunoglobulins or fragments thereof which are active in the gut. According to one aspect of this invention the food product or pharmaceutical preparation comprises a delivery system for delivering the aforementioned antibodies to the GIT wherein the delivery system is a micro-organism and the immunoglobulins are llama derived antibodies or fragments thereof. We have surprisingly found that these transformed micro-organisms will express llama heavy chain antibodies or fragments thereof on their surface and are able to reduce the viral load, normalize the pathology and mitigate the diarrhoea in an animal model of rotavirus infection. Furthermore, the llama heavy chain antibodies or fragments thereof were found to be very effective in reducing infection both in in vitro and in vivo models of rotavirus infection. Llama VHH antibody fragments have surprisingly been found to reduce the viral load, normalize the pathology and mitigate diarrhoea during rotavirus infection.

Particularly preferred LLama derived VHH sequences having affinity of rotavirus are provided by this specification in the sequence listing, SEQ ID No's 1 to 21, and FIG. 12. Alternatively, VHH sequences having at least 70%, 80%, 85%, 90%, 95%, 98% or 99% amino acid identity with SEQ ID No. 1 and having affinity for a rotavirus particle or antigen are also preferred embodiments according to this invention. VHH sequences may be derived from camellids, via immunization and/or by screening for affinity, but may also be derived from other mammalian species such as mice or humans and/or be camelized by amino acid substitutions, as described in the art. In another embodiment, the VHH sequences may be fused to yield multimeric units of 2, 3, 4, 5 or more VHH units, optionally linked via a spacer molecule. In another embodiment, several VHH sequences may be combined, either separately or in one multimeric molecule. Preferably the VHH sequences have different specificities, for instance VHH sequences may be combined to provide a wide spectrum of affinities for a particular pathogen. In a highly preferred embodiment, 2, 3, 4, 5 or more VHH sequences having affinity for any one of rotavirus strains Wa, CK5, Wa1, RRV or CK5, may be combined, as separate monomeric units or as combined units on a carrier, for instance on a probiotic bacterium and/or on a multimeric molecule.

Furthermore, llama heavy chain antibodies have also unexpectedly been found to be suitable for administration in the GIT. Llama heavy chain antibodies were found to be highly resistant to protease degradation in the stomach and to withstand the acidic environment of the stomach. This is despite the fact that the proteolytic system in the GIT is more aggressive an environment than, for example encountered in the mouth. Activity in the gut is hampered by proteolytic activity, including protease and peptidase. We have now found that even more surprisingly the in vivo production or release of antibody fragments locally in the GIT circumvents the practical problem of degradation of orally administered antibodies in the stomach and gut. The present invention is the first system which enables expression of antibodies in the GIT which are suitable for the management of rotavirus infection.

When probiotic micro-organisms are chosen as the delivery system, we have found that these transformed microorganisms will express llama heavy chain antibodies or fragments thereof on their surface and are able to reduce the viral load, normalize the pathology and mitigate the diarrhoea in an animal model of rotavirus infection.

The llama heavy chain antibodies are then expressed by the micro-organism in the GIT. Expression of the llama derived VHH antibody fragment may be both on the surface of the micro-organism and/or as a secreted protein of the micro-organism.

Preferably secreted forms of the VHH antibody fragment is in multimeric form to enhance aggregation and clearance of the viral load.

Preferably, the micro-organism or more preferably a probiotic bacterium is transformed with an expression vector comprising the gene for the llama heavy chain antibody or fragments thereof. The expression vector may contain a constitutive promoter in order to express the antibodies or fragments thereof. Such a constitutive promoter will support in situ expression of antibodies by transformed lactobacilli persisting (at least for a short period) in the intestinal tract after administration. Alternatively, the promoter may be chosen to be active only in the GIT and/or stomach/gut i.e. suitable for GIT specific expression only. This will ensure expression and/or secretion of the llama heavy chain antibody or fragments thereof in the GIT, preferably the gut. Many constitutive promoters for lactobacilli are known in the art and an example of a promoter that is specifically inducible in the GIT is Pldh (Pouwels et al “Lactobacilli as vehicles for targeting antigens to mucosal tissues by surface exposition of foreign antigens” Methods in Enzymology (2001) 336:369-389).

The expression vectors described in the examples are able to replicate in the transformed lactobacilli and express the antibodies of fragments thereof. It will be understood that the present invention is not limited to these replication expression vectors only. The whole expression cassette can be inserted in a so-called “integration” plasmid, whereby the expression cassette will be integrated into the chromosome of the lactobacilli after transformation, as known in the art (Pouwels, P. H. and Chaillou, S. Gene expression in lactobacilli (2003) Genetics of lactic acid bacteria page 143-188).

ii) Probiotic Micro-Organisms Independent from the Antibodies or Antibody Fragments.

The food product or pharmaceutical preparation according to the invention further comprises a probiotic micro-organism which is independent from the antibodies or fragments thereof.

The probiotic micro-organism may be used in either a viable or non-viable condition as desired. If the micro-organisms are to be used in a non-viable state then they may be rendered non-viable by any suitable means.

The probiotic micro-organism may be any suitable, edible, probiotic bacteria, mould or yeast and in particular may be of any of the types, including the preferred types, listed hereinabove for the micro-organism which forms a part of any delivery system for the antibodies or fragments thereof. One particularly preferred probiotic bacteria for use as the ‘independent probiotic micro-organism’ is Lactobacillus reutarii.

Advantageously the amount of the micro-organism in the delivery system in food products of the invention is between 10⁶ and 10¹¹ per serving or (for example if serving size is not known) between 10⁶ and 10¹¹ per 100 g of product, more preferred these levels are from 10⁸ to 10⁹ per serving or per 100 g of product. In some circumstances, it is advantageous of the total amount of micro-organism in the food product (i.e. the total of the amount of the micro-organism in the delivery system and the amount of the probiotic micro-organism which is independent from the antibodies or fragments thereof) is between 10⁶ and 10¹¹ per serving or (for example if serving size is not known) between 10⁶ and 10¹¹ per 100 g of product, more preferred these levels are from 10⁸ to 10⁹ per serving or per 100 g of product.

The probiotic microorganism may be added by any suitable means to the food product or pharmaceutical preparation.

### Food Products

Several food products may be prepared according to the invention, for example meal replacers, soups, noodles, ice-cream, sauces, dressing, spreads, snacks; cereals, beverages, bread, biscuits, other bakery products, sweets, bars, chocolate, chewing gum, diary products, dietetic products e.g. slimming products or meal replacers etc. For some applications food products of the invention may also be dietary supplements, although the application in food products of the above type is preferred.

In all applications the transformed micro-organisms can be added as viable cultured (wet) biomass or as a dried preparation, still containing viable micro-organisms as known in the art.

Table 1 indicates a number of products, which may be prepared according to the invention, and a typical serving size.

TABLE 1

| Product | Serving Size | |
|---|---|---|
| margarine | 15 | g |
| ice-cream | 150 | g |
| dressing | 30 | g |
| sweet | 10 | g |
| bar | 75 | g |
| meal replacer drink | 330 | ml |
| beverages | 200 | ml |

An alternative means of administration of the antibodies or fragments thereof (including a delivery system comprising a micro-organism transformed with antibodies or functional fragments thereof) and the probiotic micro-organisms comprises a dispensing implement for use with a food product comprising probiotic micro-organisms which implement is coated on at least one surface with antibodies or anti-body fragments which are active in the gut. It is preferred that the antibodies or antibody fragments comprise a delivery system for delivering the antibodies or antibody fragments to the GIT.

Yet another alternative means of administration of the antibodies or fragments thereof and the probiotic micro-organisms comprises a dispensing implement for use with a food product wherein the dispensing implement is coated on at least one surface with antibodies or anti-body fragments which are active in the gut and probiotic micro-organisms. It is preferred that the antibodies or antibody fragments comprise a delivery system for delivering the antibodies or antibody fragments to the GIT.

For both of the above alternative means of administration it is preferred that the delivery system comprises encapsulated antibodies or antibody fragments and/or that wherein the delivery system comprises a micro-organism transformed to be able to produce antibodies or fragments thereof.

The term dispensing implement covers tube, straws, knives, forks, spoons or sticks or other implements which are used to deliver a liquid or semi-solid food product to a consumer. The dispensing implement may also be used to deliver a solid food product to a consumer. This dispensing tube or straw is especially suitable for use with certain beverages where high or low pH and/or temperature means that direct addition of the micro-organism to the beverage is not recommended.

The dispensing implement can be also be used when the delivery system of the invention comprises encapsulated antibodies or fragments thereof or even with antibodies or fragments thereof per se.

After the dispensing implement is coated with the relevant components according to the above, the implement is stored in an outer envelope which is impermeable to moisture and other contamination. The coating material which contains these particles is non-toxic to humans and to bacteria and can be an oil such as corn oil or a wax. This aspect is described in U.S. Pat. No. 6,283,294 B1. Once the dispensing implement containing these components penetrates the beverage or semi-solid food product, the particles are integrated into the food product, giving a desirable dose of the antibodies or fragments thereof and the probiotic micro-organisms with a serving of the product.

Preferably, the components above to be coated onto the implement may be suspended in water which is then applied to the dispensing implement and evaporated. By using this method the dispensing implement will have a coating of the components which can then be released when the dispensing implement comes into contact with the liquid or semi-solid food product.

A still further embodiment of the invention relates to a method for making a food product or pharmaceutical preparation according to the fourth aspect of the invention.

If it is desired that the micro-organism and/or the probiotic micro-organism is/are alive in the product, for example, if the product is heated during processing, the micro-organism has to be added after the heating step (post-dosing). However, if a product is fermented with the micro-organism, a heating step after the fermentation may not be acceptable. If the product is a liquid product, administration of the micro-organism could take place by use of a dispensing implement such as a drinking straw.

A further embodiment of the invention relates to the use of the food product or pharmaceutical preparation according to the invention to deliver health benefits to the gut of a subject after administration. Such health benefits include the specific health benefit the antibody may provide. The micro-organism itself used in any delivery system may also provide several health effects for example relating to gut well being such as IBS (Irritable Bowel Syndrome), reduction of lactose maldigestion, clinical symptoms of diarrhoea, immune modulation, anti-tumor activity, adjuvant effects and enhancement of mineral uptake.

The food product or pharmaceutical preparation according to the present invention may be suitable for the management, including treatment or prophylaxis of infections caused by enteropathogenic bacteria or viruses. Other antibodies which may be incorporated into the invention will be able to provide a multitude of other health benefits.

The present invention is based on the finding that heavy chain immunoglobulins or fragments thereof of the VHH- or VNAR-type, or domain antibodies (dAbs) of the heavy or light chains of immunoglobulins or fragments thereof, of the invention may be used in the therapy or prophylaxis of infection by enteropathogenic micro-organisms. Furthermore, the immunoglobulins or fragments thereof of the VHH- or VNAR-type, or domain antibodies (dAbs) of the heavy or light chains of immunoglobulins or fragments thereof, may be used in the therapy or prophylaxis of viral gastroenteritis or diarohoea caused by the enteropathogenic microorganism rotavirus.

A further advantage of the present invention is that the use of food products or pharmaceutical preparations comprising probiotic microorganisms-expressing immunoglobulins or fragments thereof of the VHH- or VNAR-type, or domain antibodies (dAbs) of the heavy or light chains of immunoglobulins or fragments thereof, enables the micro-organism used as part of any delivery system, for example Lactobacillus, to provide the normal health benefits associated therewith, together with the prophylactic/therapeutic benefits in the management of the infection to be treated. This “dual effect” therapy provides greater health benefits to the subject than that known in the art.

In accordance with another embodiment of the present invention, the heavy chain immunoglobulins or fragments thereof of the VHH type are derived from camelids, including llama and camels. Many llama derived heavy chain antibody fragments have been disclosed in the art. More preferred is the heavy chain immunoglobulin or fragment thereof which shows binding affinity with a dissociation constant of at least 10 exp 5 for rotavirus, especially rotavirus strains Wa, CK5, Wa1, RRV or CK5.

We have surprisingly found that llama heavy chain antibodies are effective in the management of rotavirus infection. When the antibodies used are llama heavy chain antibodies, the health benefit delivered will include an anti-diarhoeal effect. Hence, llama heavy chain antibodies can be used in the management of rotavirus infection, including the therapy or prophylaxis of rotavirus infection. We have found that llama VHH antibody fragments can reduce the viral load, normalize the pathology and mitigate diarrhoea during rotavirus infection. Rotavirus continues to be the single most common cause of infantile diarrhoea in the world and most children get infected during the first 5 years of life. In developing countries, rotavirus induced diarrhoea may cause 600,000 to 870,000 deaths each year and in developed countries, rotavirus disease accounts for immense economic loss.

Furthermore, llama heavy chain antibodies have also unexpectedly been found to be suitable for administration in the gut. We have surprisingly found that the llama heavy chain antibodies were found to be highly resistant to protease degradation in the stomach and to withstand the acidic environment of the stomach. This is despite the fact that the proteolytic system in the gut/stomach is more aggressive an environment than, for example encountered in the mouth. The in vivo production of antibody fragments locally in the GIT circumvents the practical problem of degradation of orally administered antibodies in the stomach. The present invention is the first system which enables expression of antibodies in the GIT which are suitable for the management of rotavirus infection.

Hence, the use of food products or pharmaceutical preparations comprising lactobacilli expressing llama heavy chain antibodies enables the lactobacilli used as part of any delivery system to provide the normal health benefits associated therewith together with the prophylactic/therapeutic benefits in the management of rotavirus infection. The present invention is the first system which enables expression of antibodies in the GIT which are suitable for the management of rotavirus infection.

It will be understood that the food product or pharmaceutical preparation can be administered in order to deliver a health benefit to the subject and/or to combat a specific disease or infection. The choice of the antibody will depend on the disease to be treated.

Preferably, the micro-organism is transformed with an expression vector comprising the gene for the llama heavy chain antibody or fragment thereof. Either an integrating or a replicating vector may be used.

If encapsulation is chosen as the delivery system, the encapsulation method should survive passage to the stomach through the GIT and should be able to provide a sustained release of the antibody over a set period of time. This will ensure that the llama heavy chain antibody or fragment is delivered over time to the stomach. Llama heavy chain antibodies or heavy chains thereof are particularly suitable for this encapsulation method due to their ability to survive in the gut when released.

Specifically, the antibodies which form part of any delivery system may be delivered to the GIT using a micro-organism transformed with llama heavy chain antibodies comprising the steps of i) transforming the micro-organism with the gene encoding llama heavy chain antibodies; and ii) administering the transformed micro-organism to the GIT of the human or animal in need of therapy.

The invention will now be further illustrated by the description of suitable embodiments of the preferred food products for use in the invention. It is believed to be well within the ability of the skilled person to use the teaching provided therewith to prepare other products of the invention.

### Margarines and Other Spreads

Typically these are oil in water or water in oil emulsions, also spreads which are substantially fat free are covered. Typically these products are spreadable and not pourable at the temperature of use e.g. 2-10 C. Fat levels may vary in a wide range e.g. full fat margarines with 60-90 wt % of fat, medium fat margarines with 30-60 wt % of fat, low fat products with 10-30 wt % of fat and very low or fat free margarines with 0 to 10 wt % of fat.

The fat in the margarine or other spread may be any edible fat, often use are soybean oil, rapeseed oil, sunflower oil and palm oil. Fats may be used as such or in modified form e.g. hydrogenated, esterified, refined etc. Other suitable oils are well known in the art and may be selected as desired.

The pH of a margarine or spread may advantageously be from 4.5 to 6.5. Examples of spreads other than margarines are cheese spreads, sweet spreads, yoghurt spreads etc.

Optional further ingredients of spreads may be emulsifiers, colourants, vitamins, preservatives, emulsifiers, gums, thickeners etc. The balance of the product will normally be water.

A typical size for an average serving of margarine or other spreads is 15 grams. Preferred VHH-producing Lactobacillus in the margarine or spread are 10⁶ and 10¹¹ per serving most preferred 10⁸ to 10¹⁰ per serving. The Lactobacillus strain has to be added aseptically after the heating steps in the process. Alternatively, encapsulated VHH's may be added to these food products. Preferably between 25 and 5000 μg per serving is added, more preferably between 50 and 500 μg are added per serving. Most preferably two or three servings are given each day.

### Frozen Confectionary Products

For the purpose of the invention the term frozen confectionery product includes milk containing frozen confections such as ice-cream, frozen yoghurt, sherbet, sorbet, ice milk and frozen custard, water-ices, granitas and frozen fruit purees.

Preferably the level of solids in the frozen confection (e.g. sugar, fat, flavouring etc) is more than 3 wt %, more preferred from 10 to 70 wt %, for example 40 to 70 wt %.

Ice-cream will typically comprise 2 to 20 wt % of fat, 0 to 20 wt % of sweeteners, 2 to 20 wt % of non-fat milk components and optional components such as emulsifiers, stabilisers, preservatives, flavouring ingredients, vitamins, minerals, etc, the balance being water. Typically ice-cream will be aerated e.g. to an overrun of 20 to 400%, more general 40 to 200% and frozen to a temperature of from −2 to −200 C, more general −10 to −30 C. Ice-cream normally comprises calcium at a level of about 0.1 wt %.

A typical size of an average serving of frozen confectionary material is 150 grams. Preferred Lactobacillus levels are from 10⁶ and 10¹¹ per serving, more preferred these levels are from 10⁷ to 10¹⁰ per serving most preferred 10⁸ to 10⁹ per serving. The Lactobacillus strain has to be added aseptically after the heating steps in the process. Alternatively, encapsulated VHH's may be added to these food products. Preferably between 25 and 5000 μg per serving is added, more preferably between 50 and 500 μg are added per serving. Most preferably two or three servings are given each day.

Beverages, for example Tea Based Products or Meal Replacers

Lactobacillus can advantageously be used to beverages for example fruit juice, soft drinks etc. A very advantageous beverage in accordance to the invention is a tea based product or a meal replacers drink. These products will be described in more detail herein below. It will be apparent that similar levels and compositions apply to other beverages comprising vitamin Lactobacillus bacteria.

For the purpose of this invention the term tea based products refers to products containing tea or tea replacing herbal compositions e.g. tea-bags, leaf tea, herbal tea bags, herbal infusions, powdered tea, powdered herbal tea, ice-tea, ice herbal tea, carbonated ice tea, carbonated herbal infusion etc.

Typically some tea based products of the invention may need a preparation step shortly before consuming, e.g. the making of tea brew from tea-bags, leaf tea, herbal tea bags or herbal infusions or the solubilisation of powdered tea or powdered herbal tea. For these products it is preferred to adjust the level of Lactobacillus in the product such that one serving of the final product to be consumed has the desired levels of Lactobacillus as described above.

For ice-tea, ice herbal tea, carbonated ice tea, carbonated herbal infusions the typical size of one serving will be 200 ml or 200 grams.

Meal replacer drinks are typically based on a liquid base which may for example be thickened by means of gums or fibres and whereto a cocktail of minerals and vitamins are added. The drink can be flavoured to the desired taste e.g. fruit or choco flavour. A typical serving size may be 330 ml or 330 grams.

Both for tea based beverages and for meal replacer drinks, preferred Lactobacillus levels are 10⁶ and 10¹¹ per serving, more preferred these levels are form 10⁷ to 10¹⁰ per serving most preferred 10⁸ to 10⁹ per serving. Alternatively, encapsulated VHH's may be added to these food products. Preferably between 25 and 5000 μg per serving is added, more preferably between 50 and 500 μg are added per serving. Most preferably two or three servings are given each day.

For products which are extracted to obtain the final product, generally the aim is to ensure that one serving of 200 ml or 200 grams comprises the desired amounts as indicated above. In this context, it should be appreciated that normally only part of the Lactobacillus present in the tea based product to be extracted will eventually be extracted into the final tea drink. To compensate for this effect generally it is desirable to incorporate into the products to be extracted about 2 times the amount as is desired to have in the extract.

For leaf tea or tea-bags typically 1-5 grams of tea would be used to prepare a single serving of 200 mls.

If tea-bags are used, the Lactobacillus may advantageously be incorporated into the tea component. However it will be appreciated that for some application it may be advantageous to separate the Lactobacillus from the tea, for example by incorporating it into a separate compartment of the tea bag or applying it onto the tea-bag paper. Alternatively, the micro-organism may be administered in dried form through the use of a straw, spoon or stick with a coating of dried microorganism.

### Salad Dressings or Mayonnaise

Generally dressings or mayonnaise are oil in water emulsions, the oil phase of the emulsion generally is 0 to 80 wt % of the product. For non fat reduced products the level of fat is typically from 60 to 80%, for salad dressings the level of fat is generally 10-60 wt %, more preferred 1540 wt %, low or no fat dressings may for example contain triglyceride levels of 0, 5, 10, 15% by weight.

Dressings and mayonnaise are generally low pH products having a preferred pH of from 2-6.

Dressings or mayonnaise optionally may contain other ingredients such as emulsifiers (for example egg-yolk), stabilisers, acidifiers, biopolymers, bulking agents, flavours, colouring agents etc. The balance of the composition is water which could advantageously be present at a level of 0.1 to 99.9 wt %, more general 20-99 wt %, most preferred 50 to 98 wt %.

A typical size for an average serving of dressings or mayonnaise is 30 grams. Preferred levels of Lactobacillus in such products would be 10⁶ and 10¹¹ per serving, more preferred these levels are from 10⁷ to 10¹⁰ per serving most preferred 10⁸ to 10⁹ per serving. The Lactobacillus strain has to be added aseptically after the heating steps in the process. Alternatively, encapsulated VHH's may be added to these food products. Preferably between 25 and 5000 μg per serving is added, more preferably between 50 and 500 μg are added per serving. Most preferably two or three servings are given each day.

### Meal Replacer Snacks or Bars

These products often comprise a matrix of edible material wherein the Lactobacillus can be incorporated. For example the matrix may be a fat based (e.g. couverture or chocolate) or may be based on bakery products (bread, dough, cookies etc) or may be based on agglomerated particles (rice, grain, nuts, raisins, fruit particles).

A typical size for a snack or meal replacement bar could be 20 to 200 g, generally from 40 to 100 g. Preferred levels of Lactobacillus in such products would be 10⁶ and 10¹¹ per serving, more preferred these levels are from 10⁷ to 10¹⁰ per serving most preferred 10⁸ to 10¹⁰ per serving. The Lactobacillus strain has to be added aseptically after the heating steps in the process. Alternatively, encapsulated VHH's may be added to these food products. Preferably between 25 and 5000 μg per serving is added, more preferably between 50 and 500 μg are added per serving. Most preferably two or three servings are given each day.

Further ingredients may be added to the above product such as flavouring materials, vitamins, minerals etc.

For each of the above food products, the amount of Lactobacillus per serving has been given as a preferred example. It will be understood that alternatively any suitable micro-organism or virus may be present at this level.

### Lemonade Powder

Lactobacillus can also be used in dry powders in sachets, to be dissolved instantly in water to give a refreshing lemonade. Such a powder may have a food-based carrier, such as maltodextrin or any other. Optional further ingredients may be colourants, vitamins, minerals, preservatives, gums, thickeners etc.

A typical size for an average serving or margarine or other spreads is 30-50 grams. Preferred VHH-producing Lactobacillus in the lemonade powder are 10⁶ and 10¹¹ per serving most preferred 10⁸ to 10¹⁰ per serving. The Lactobacillus strain has to be sprayed on the carrier in such a way that it is kept alive, according to methods known by those skilled in the art. Alternatively, encapsulated VHH's may be added to these food products. Preferably between 25 and 5000 μg per serving is added, more preferably between 50 and 500 μg are added per serving.

In all the above products the transformed micro-organism can be added as viable cultured (wet) biomass or as a dried preparation, still containing the viable micro-organisms as known in the art.

The invention will be further illustrated in the examples.

### EXAMPLES

### Examples 1 to 3

### Generation of Antibody Fragments with Subsequent In-Vitro and in Vivo Testing

### Example 1

Selection of rotavirus specific heavy-chain antibody fragments from a llama immune phage display library and production in yeast.

Rhesus rotavirus strain RRV (serotype G3) was purified, amplified and concentrated as described previously (Svensson L., Finlay B. B., Bass D., Vonbonsdorff C. H., Greenberg H. B. “Symmetrical infection on polarised human intestinal epithelial (CaCo-2) cells”. J. Virol. (1991) 65, 4190-4197.

A llama was immunized subcutaneously and intramuscularly at day 0, 42, 63, 97 and 153 with 5×10¹² pfu of rotavirus strain RRV.

Prior to immunization, the viral particles were taken up in oil emulsion (1:9 V/V, antigen in PBS: Specol (Bokhout, B. A., Van Gaalen, C., and Van Der Heijden, Ph. J. “A selected water-in-oil emulsion: composition and usefulness as an immunological adjuvant”. Vet. Immunol. Immunopath. (1981) 2: 491-500 and Bokhout, B. A., Bianchi, A. T. J., Van Der Heijden, Ph. J., Scholten, J. W. and Stok, W. “The influence of a water-in-oil emulsion on humoral immunity”. Comp. Immun. Microbiol. Infect. Dis. (1986) 9: 161-168. as described before (Frenken, L. G. J., et al. Isolation of antigen specific Llama V_{HH} antibody fragments and their high level secretion by Saccharomyces cerevisiae. J. Biotechnol. (2000) 78, 11-21). The immune response was followed by titration of serum samples in ELISA with RRV rotavirus coated at a titer of 4×10⁶ pfu/ml in 0.9% NaCl following the protocol described before (De Haard, 30H. J., van Neer, N., Reurs, A., Hufton, S. E., Roovers, R. C., Henderikx P., de Bruine A. P., Arends J. W., and Hoogenboom, H. R. “A large non-immunized human Fab fragment phage library that permits rapid isolation and kinetic analysis of high affinity antibodies”. J. Biol. Chem. (1999) 274: 18218-18230.; Frenken, L. G. J., et al. “Isolation of antigen specific Llama V_{HH} antibody fragments and their high level secretion by Saccharomyces cerevisiae”. J. Biotechnol. (2000) 78, 11-21).

An enriched lymphocyte population was obtained from the 153-day blood sample of about 150 ml via centrifugation on a Ficoll (Pharmacia) discontinuous gradient. From these cells, total RNA (between 250 and 400 μg) was isolated by acid guanidium thiocyanate extraction Chomczynski, P. and Sacchi, N. “Single-step method of RNA isolation by acid guanidinium thiocyanate-phenol-chloroform extraction”. Anal. Biochem. (1987)162:156-159. Subsequently, first strand cDNA was synthesized using the Amersham first strand cDNA kit (RPN1266). In a 20 μl reaction mix 0.4-1 μg mRNA was used. The 6-mer random primer was used to prime the first DNA strand. After cDNA synthesis, the reaction mix was directly used for amplification by PCR. VHH genes were amplified with primers

Lam-16: (GAGGTBCARCTGCAGGASAGYGG);
Lam-17: (GAGGTBCARCTGCAGGASTCYGG);
Lam-07 (priming to the short hinge region);
and
Lam-08 (long hinge specific)

(Frenken, L. G. J., et al. “Isolation of antigen specific Llama V_{HH} antibody fragments and their high level secretion by Saccharomyces cerevisiae” J. Biotechnol. (2000) 78, 11-21). Amplification of DNA was performed as described by De Haard, H. J., van Neer, N., Reurs, A., Hufton, S. E., Roovers, R. C., Henderikx P., de Bruine A. P., Arends J. W. and Hoogenboom H. R. “A large non-immunized human Fab fragment phage library that permits rapid isolation and kinetic analysis of high affinity antibodies”. J. Biol. Chem. (1999)274: 18218-18230.

The amplified products were digested with PstI and NotI (New England Biolabs, US) and cloned in phagemid vector pUR5071, which is based on pHEN1 (Hoogenboom, H. R., Griffiths, A. D., Johnson, K. S., Chiswell, D. J., Hudson, P. and Winter, G. “Multi-subunit proteins on the surface of filamentous phage: methodologies for displaying antibody (Fab) heavy and light chains”. Nucleic Acids Res. (1991) 19: 4133-4137) and contains a hexahistidine tail for Immobilized Affinity Chromatography (Hochuli, E., Bannwarth, W., Döbeli, H., Gentz, R. and Stüber, D. “Genetic approach to facilitate purification of recombinant proteins with a novel metal chelate adsorbent”. Bio/Technol. (1988) 6:1321-1325) and a c-myc derived tag (Munro S, and Pelham H. R. “An Hsp70-like protein in the ER: identity with the 78 kd glucose-regulated protein and immunoglobulin heavy chain binding protein”. Cell (1986) 46: 291-300) for detection. Ligation and transformation were performed as was described before (De Haard, H. J., van Neer, N., Reurs, A., Hufton, S. E., Roovers, R. C., Henderikx P., de Bruine A. P., Arends J. W. and Hoogenboom H. R. “A large non-immunized human Fab fragment phage library that permits rapid isolation and kinetic analysis of high affinity antibodies”. J. Biol. Chem. (1999) 274: 18218-18230.). The rescue with helperphage VCS-M13 and PEG precipitation was performed as described by Marks, J. D., Hoogenboom, H. R., Bonnert, T. P., McCafferty, J., Griffiths, A. D. and Winter, G. “By-passing immunization: Human antibodies from V-gene libraries displayed on phage”. J. Mol. Biol. (1991) 222: 581-597.

Selections of rotavirus specific phages were performed via the biopanning method (Marks J. D., Hoogenboom, H. R., Bonnert, T. P., McCafferty, J., Griffiths, A. D. and Winter, G. “By-passing immunization: Human antibodies from V-gene libraries displayed on phage”. J. Mol. Biol. (1991) 222: 581-597.) by coating of rotavirus strain RRV (2.5×10⁷ pfu/ml at round 1; 5×10⁴ pfu/ml at round 2; 500 pfu/ml at round 3). Immunotubes (Nunc, Roskilde, Denmark) were coated overnight at 4° C. with either a 1:1000 dilution of anti-rotavirus rabbit sera or anti-rotavirus guinea pig sera in carbonate buffer (16% (v/v) 0.2 M NaHCO₃+9% (v/v) 0.2 M Na₂CO₃). Viral particles were captured via polyclonal anti-rotavirus sera. In addition to the standard selections, the antibody fragment displaying phages have selected in an acidic environment. This was done by selecting in a dilute HCl solution (pH 2.3). After this adapted selection process, the standard procedure was followed.

Soluble VHH was produced by individual E. coli TG1 clones as described by Marks J. D., Hoogenboom, H. R., Bonnert, T. P., McCafferty, J., Griffiths, A. D. and Winter, G. “By-passing immunization: Human antibodies from V-gene libraries displayed on phage”. J. Mol. Biol. (1991) 222: 581-597. Culture supernatants were tested in ELISA. Microlon F (Greiner Bio-One GmbH, Germany) plates were coated with 50 μl/well of a 1:1000 dilution of either anti-rotavirus rabbit polyclonal sera or anti-rotavirus guinea pig polyclonal sera in carbonate buffer (16% (v/v) 0.2 M NaHCO₃+9% (v/v) 0.2 M Na₂CO₃) and subsequently incubated with rotavirus strain RRV or CK5 (approx. 10⁹ pfu/ml). After incubation of the VHH containing supernatants, VHH's were detected with a mixture of the mouse anti-myc monoclonal antibody 9E10 (500 ng/ml, in-house production) and anti-mouse HRP conjugate (250 ng/ml, Dako, Denmark). Alternatively, detection was performed with anti-6×His-HRP antibody conjugate (1000 ng/ml, Roche Molecular, US). Fingerprint analysis (Marks, J. D., Hoogenboom, H. R., Bonnert, T. P., McCafferty, J., Griffiths, A. D. and Winter, G. “By-passing immunization: Human antibodies from V-gene libraries displayed on phage”. J. Mol. Biol. (1991) 222: 581-597) with the restriction enzyme HinFI (New England Biolabs, US) was performed on all clones. Sequencing was performed at Baseclear B. V. (Leiden, The Netherlands).

A set of rotavirus-specific antibody fragments was selected. DNA sequences encoding these antibody fragments were isolated from pUR5071 (PstI/BstEII, New England Biolabs, US)) and cloned into pUR4547 which is identical to the previously described pUR4548 (Frenken, L. G. J., et al., “Isolation of antigen specific Llama V_{HH} antibody fragments and their high level secretion by Saccharomyces cerevisiae”. J. Biotechnol. (2000) 78, 11-21) but does not encode any C-terminal tag-sequences. This episomal yeast expression vector contains the GAL7 promoter, the SUC2 signal sequence for high level expression and secretion into the growth medium, respectively. The S. cerevisiae strain VWK18gal1 was transformed and induced for antibody fragment production as described previously (Van der Vaart, J. M., “Expression of VHH antibody fragments in Saccharomyces cerevisiae”. In Methods in Molecular Biology (2001) Vol. 178, p 359-366, Edited by P. M. O'Brien and R. Aiken, Humana Press Inc., Totowa, N.J.). Antibody fragments were purified and concentrated by filtration over microcon filters with a 10 kDa cut-off (Amicon, US).

### Example 2

In Vitro Inhibition of Rotavirus

Bovine Rotavirus Compton CK5 was obtained from the Moredun Institute, Midlothian, Scotland and the BS-C1 cells were purchased from the European Animal Cell Culture Collection.

The BS-C1 cells were cultured in Earles Modified Essential Medium supplemented with 10% Heat inactivated foetal calf serum, 1% MEM Amino Acids solution (100×), 20 mmol l⁻¹ L-Glutamine, 100 I.U ml⁻¹ penicillin, 100 μg ml⁻¹ streptomycin and 2.5 μg ml⁻¹ amphotericin B (all from Sigma, US).

CK5 Rotavirus stock was diluted in Serum Free Medium (SFM) EMEM supplemented with 1% MEM Amino Acids solution (100×), 20 mmol l⁻¹ L-Glutamine and 0.5 μg/ml crystalline trypsin and then 5 ml of diluted seed was added to confluent monolayers of BS-C1 cells in 162 cm² tissue culture flasks (Costar, UK). The virus was adsorbed onto the cells for one hour at 37° C. then the medium was topped up to 75 ml. The bottles were incubated at 37° C. until complete cytopathic effect was observed. Cultures were frozen (−70° C.) and thawed twice, then pooled and centrifuged at 1450 g for 15 minutes at 4° C. to remove cell debris. The supernatant was decanted and stored in aliquots at −70° C.

Monolayers of BS-C1 cells were cultured in 12-well tissue culture plates at 37° C. in an atmosphere of 95% air and 5% carbon dioxide. The medium was removed and replaced with SFM for at least 2 hours prior to use. The CK5 virus was diluted to give approximately 50 pfu/ml in SFM. The selected anti-rotavirus fragments were diluted in SFM and then equal volumes of virus and fragment dilution were mixed (200 μl total volume) and incubated for one hour at 37° C.

The virus-fragment mixtures were then plated onto the monolayers of cells (three replicate wells each). The plates were incubated for one hour at 37° C. in an atmosphere of 95% air and 5% carbon dioxide. Subsequently, the virus was removed and an overlay consisting of 0.75% Sea Plaque Agarose (FMC) in EMEM containing 100 I.U. ml⁻¹ penicillin, 100 μg ml⁻¹ streptomycin, 2.5 μg ml⁻¹ amphotericin B and 0.1 μg/ml crystalline trypsin was added. Plates were then incubated at 37° C. in an atmosphere of 95% air and 5% carbon dioxide for 4 days. After fixing and staining with 1% crystal violet in 10% formaldehyde, the agarose was removed, the wells washed with water and the plaques counted.

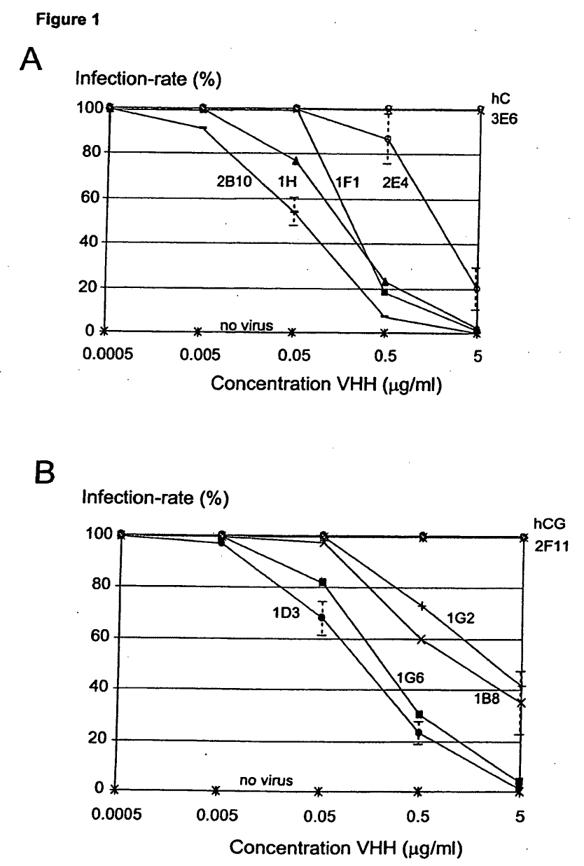

From 23 clones tested according to the method describe above, nine produced antibody fragments capable of neutralising this rotavirus strain. Fragments 2B10 and 1D3 most effectively neutralised rotavirus in the plaque assays (FIG. 1)

FIG. 1 shows virus neutralisation of rotavirus CK5 determined by an in vitro plaque assay. The average neutralisation-rate from the four obtained measurements is indicated at each data-point. If there is a spread of over 10% at a data point, the two most extreme measurements are indicated (dashed bar). The tested antibody fragments were divided over 2 graphs, A and B. A's negative controls either the virus was omitted (no virus) or a non-rotavirus specific VHH was added. The non-specific VHH fragment is specific for the human pregnancy hormone hCG. Isolation of this fragment has been described (Spinelli, S., Frenken, L., Bourgeois, D., de Ron, L., Bos, W., Verrips, T., Anguille, C., Cambillau, C. and Tegoni, M. “The crystal structure of a llama heavy chain domain”. Nat. Struct. Biol. (1996) 3: 752-757; Frenken, L. G. J., et al. “Isolation of antigen specific Llama V_{HH} antibody fragments and their high level secretion by Saccharomyces cerevisiae”. J. Biotechnol. (2000) 78, 11-21).

Hence, using this method, a number of VHH fragments were identified that can inhibit rotavirus infection in an in vitro system.

### Example 3

### In Vivo Rotavirus Inhibition in Mice

Some of the VHH fragments selected via the approach described in example 2 were used in in vivo experiments to study the efficacy of these antibody fragment in the prevention or treatment of rotavirus induced diarrhoea in mice. This model system has been frequently used for study of rotavirus infection (Ebina, T, Ohta, M, Kanamaru, Y. Yamamotoosumi, Y. Baba, K. “Passive immunisations of suckling mice and infants with bovine colostrum containing antibodies to human rotavirus”. J Med Virol. (1992) 38: 117-123).

14 days pregnant, rotavirus negative BALB/c mice were obtained from Mölleg\dot{a}rd, Denmark. The mice were housed individually in the animal facility at Huddinge Hospital. Approval was obtained from the local ethical committee of Karolinska Institute at Huddinge Hospital, Sweden. Normal pellet diet and water was provided ad libitum.

In order to examine whether the fragments can inhibit infection when bound to rotavirus (RV), selected VHH fragments were premixed with titrated amounts (2×10⁷ ffu) of RRV before it was used for infection on day 1.

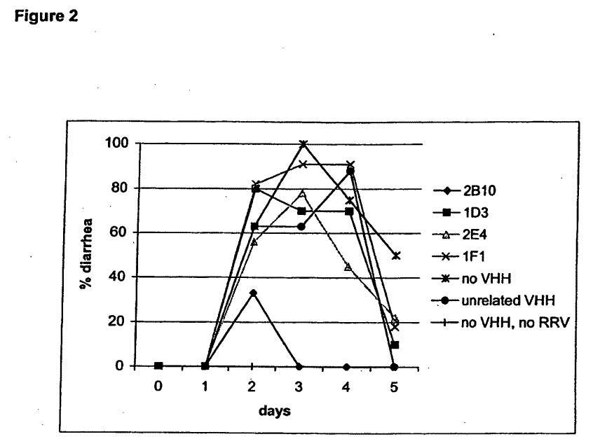

Four-day old mice pups were treated daily with VHH fragments, including day 0 (day before infection) up to and including day 4 (FIG. 2) and diarrhoea was assessed. A marked reduction in occurrence of diarrhoea was observed for antibody fragment 2B10, shown in FIG. 2. The number of pups with diarrhoea is significantly lower at day 2 in the group receiving fragment 2B10 compared to the untreated group. Moreover, at days 3, 4 and 5 none of the pups in 2B10 treated group displayed signs of diarrhoea compared to the majority of the pups in the other RRV treated groups (FIG. 2). No statistically significant effects of the unrelated VHH fragment RR6 (directed against azo dyes; Frenken, L. G. J., et al. Isolation of antigen specific Llama V_{HH} antibody fragments and their high level secretion by Saccharomyces cerevisiae”. J. Biotechnol. (2000) 78, 11-21) was found compared to the untreated group.

Additionally, the mean number of diarrhoea days per mouse pup were calculated for each treatment group as the number of diarrhoea days per pup divided by the total number of pups per treatment group. For the fragment 2B10 treated groups this was found to be significantly reduced to 0.33±0.21 days compared to 2.87±0.29 days for the untreated group.

It is important to note that from now on in the following examples the pUR5071 derived plasmid containing the gene encoding for fragment 2B10 was named pUR655 and the encoded antibody was renamed fragment VHH1 (Peter Pouwels et al “Lactobacilli as vehicles for targeting antigens to mucosal tissues by surface exposition of foreign antigens”).

### Example 4 (a to k)

Further similar experiments separate to Examples 1 to 3 were carried out as follows:

a) Construction of Anti-Rotavirus scFv-2A10 and VHH1 Expression Vectors.

Total RNA was extracted from an anti-rotavirus mAb 2A10 (IgA class) secreting hybridoma (Giammarioli et al. (1996) Virol. 225:97-110)). Variable region encoding sequences of both the heavy (VH) and light (VK) chains were amplified using a 5′ RACE kit (5′ RACE System for Rapid Amplification of cDNA Ends (Version 2.0, Invitrogen™ life technologies, Carlsbad, Calif.). The primers for the 5′ RACE of VH were

ACRACE1: 5′-CAGACTCAGGATGGGTAAC-3′,
ACRACE2: 5′-CACTTGAATGATGCGCCACTGTT-3′,
ACRACE3: 5′-GAGGGCTCCCAGGTGAAGAC-3′,
while the primers,
mkRACE1 (5′-TCATGCTGTAGGTGCTGTCT-3′),
mkRACE2 (5′-TCGTTCACTGCCATCAATCT-3′)
and
mkRACE3 (5′-TGGATGGTGGGAAGATGGAT-3′)

were utilized to amplify the variable region of the light chain. The resulting A-tailed PCR product was cloned into a pGEM®-T easy vector with 3′-T overhangs and sequenced. The VH and VK sequences were fused together with a linker encoding gene (with the amino acid sequence (G₄S)₃). Both chains were re-amplified from the cloned 5′ RACE Products using the primers

ClaI-VHs
(5′-TTTTATCGATGTGCAGTTGGTGGAGTCTGG-3′)
and
Linker-VHas
(5′-CGATCCGCCACCGCCAGAGCCACCTCCGCCTGAACCGCCTCCACCT
GAGGAGACGGTGACCGTGG-3′);
Linker-VKs
(5′-GGTGGAGGCGGTTCAGGCGGAGGTGGCTCTGGCGGTGGCGGATCGG
ACATTGTGATGACCCAGTC-3′)
and
EcoRI-Vkas
(5′-TTTTGAATTCTTTTATTTCCA GCCTGGTCC-3′).

The resulting VH and VK PCR products were mixed together and used as a template for a fusion PCR using the primers ClaI-VHs and EcoRI-VKas. The fused PCR products were cloned into a pGEM®-T easy vector after addition of overhang A using Taq DNA polymerase. The fused scFv-2A10 encoding sequence was finally cut out from the plasmids using EcoRI plus ClaI and subcloned into pBluescript II SK (+) (Stratagene, La Jolla, Calif.) containing an E-tag (pBS-E-tag).

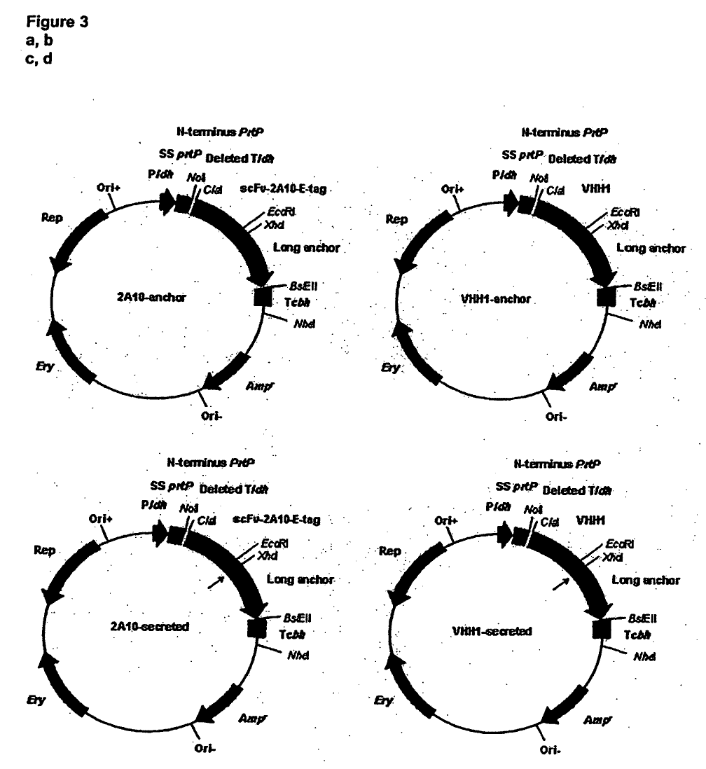

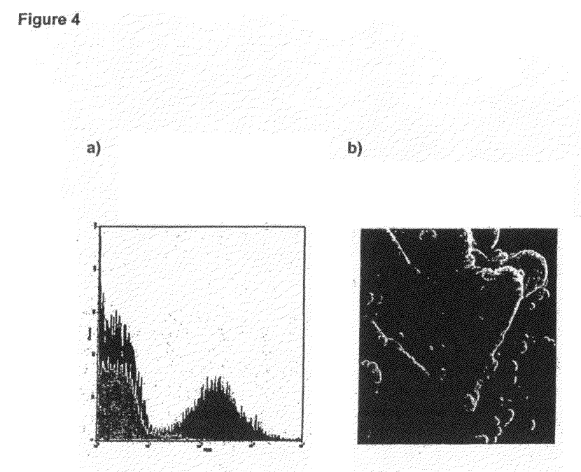

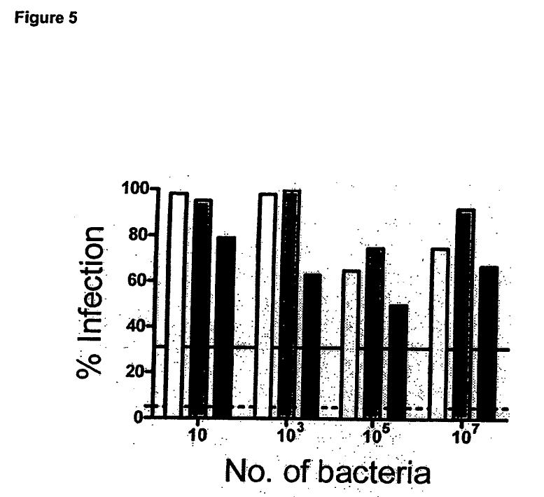

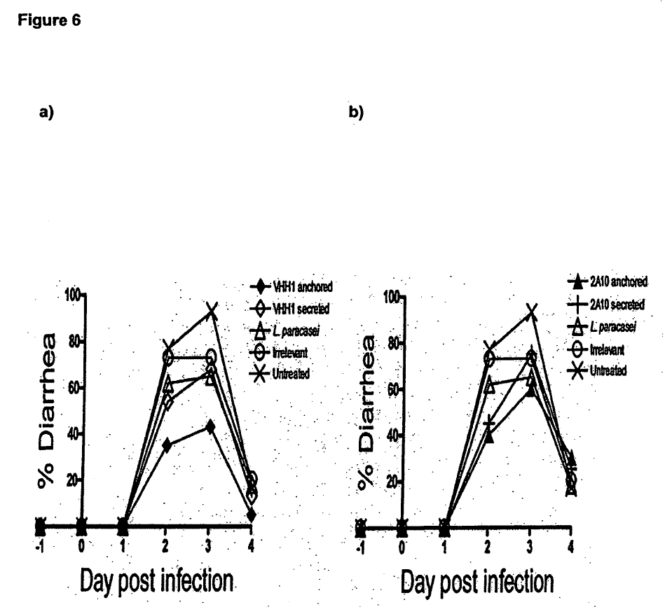

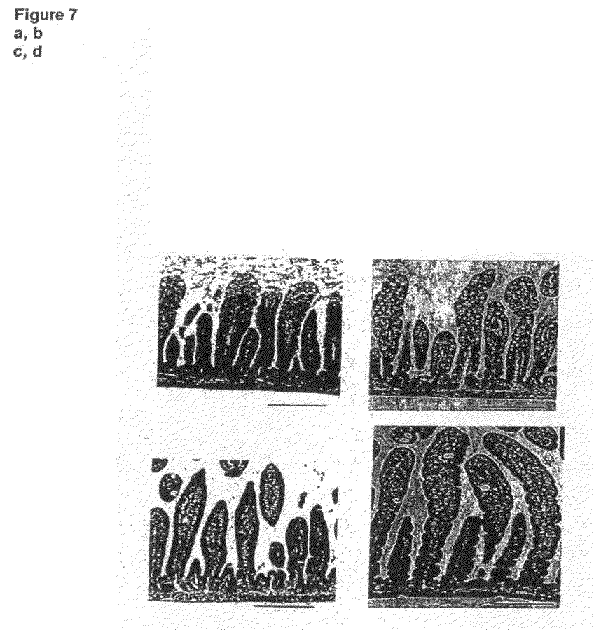

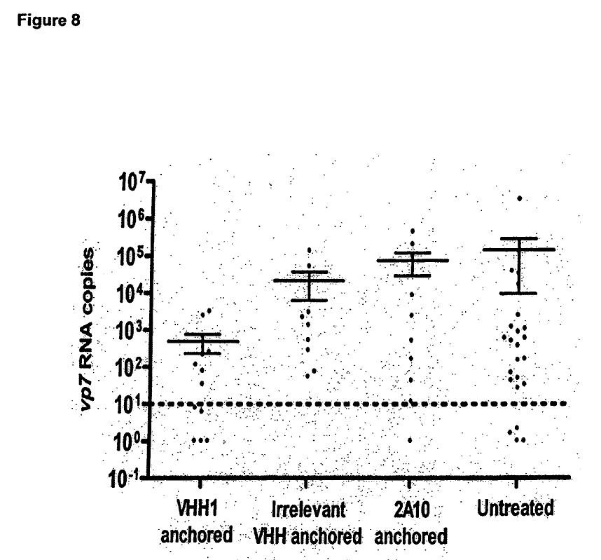

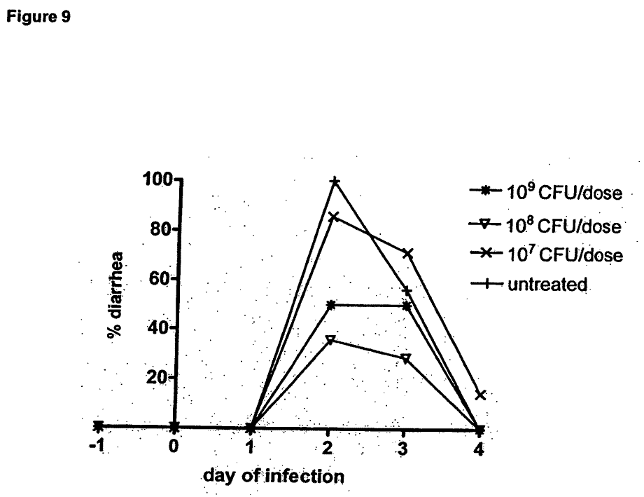

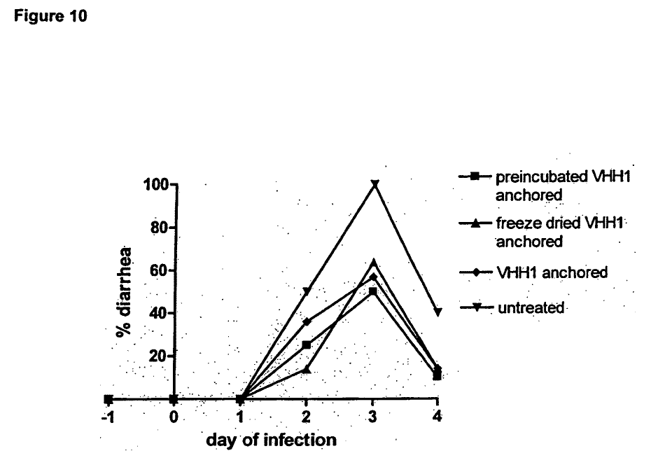

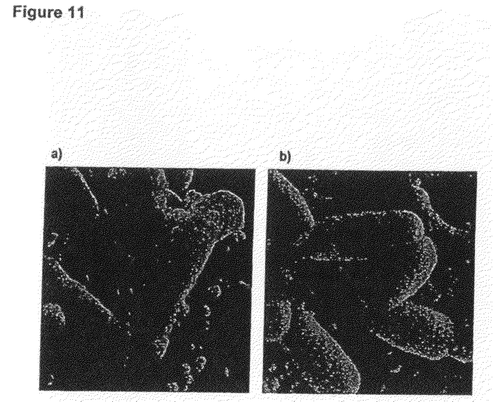

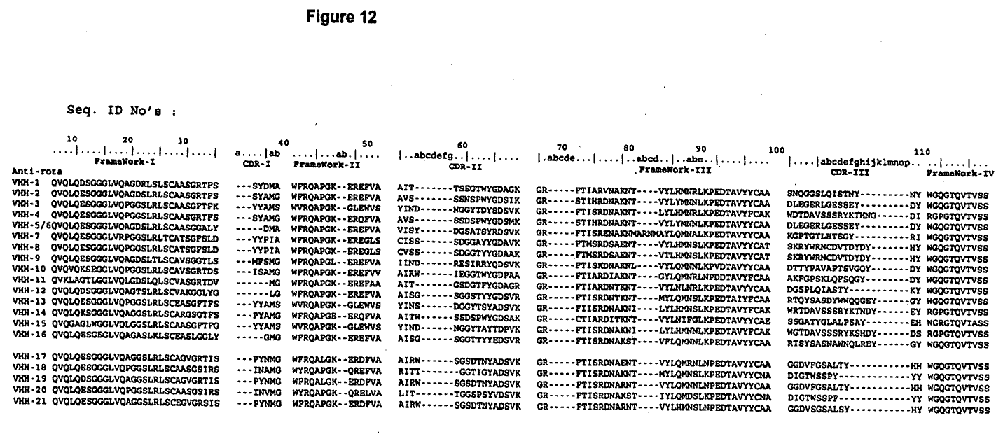

The VHH1 was amplified from pUR655 using a sense primer containing ClaI restriction site and an anti-sense primer containing EcoRI restriction site and then inserted into the pBS-E-tag vector. For generation of surface expressed antibody fragments, the scFv-2A10-E-tag and the VHH1-E-tag were excised from the pBS-E-tag vector using ClaI and XhoI restriction sites, and fused to an anchor sequence, the last 244 amino acids of the proteinase P protein of L. casei (Krüger et al, Nature Biotechnol (2002) 20:702-706), into the Lactobacillus expression vector pLP502 (FIGS. 3A and 3C). To generate the secreted antibody fragment, a stop codon (TAA) was inserted by PCR amplification after the E-tag and the products were inserted into pLP502 between the ClaI and XhoI restriction sites (FIGS. 3B and 3D). The pLP502 vector contains the constitutive promoter of the lactate dehydrogenase gene (Pldh). (Pouwels et al. “Lactobacilli as vehicles for targeting antigens to mucosal tissues by surface exposition of foreign antigens” Methods in Enzymology (2001) 336:369-389). Transformation into L. paracasei was performed as previously described (Kruger et a/(2002 as above).

### b) Comparison of Expression Levels of Antibody Specific Transgenes in Transformed Lactobacilli.

Total RNA was extracted from different lactobacilli constructs cultured to an OD₆₀₀ of 0.8 (QIAGEN) and reverse transcription was performed after digesting residual DNA with RQ1 DNase (Promega). The amount of mRNA for the different antibody fragments was measured using the qPCR™ core kit for SYBR® green I (MedProbe, Oslo, Norway) and ABI PRISM 7000 sequence detection system (PE Applied Biosystems, Foster City, Calif.). Typical profile times used were: initial step, 95° C., 10 minutes followed by a second step, 95° C. 15 seconds and 58° C. 1 minute, 40 cycles. Pooled cDNA was used for the generation of a standard curve for 16SrRNA and the antibody insert using the primers

p0 (5′GAGAGTTTGATCCTGGCTCAG 3′)
and
p6 (5′CTACGGCTACCTTGTTACGA 3′)
for the 16SrRNA and
primers prtpsp (5′TCTTGCCAGTCGGCGAAAT 3′)
and
XhoI-VHH (5′CCGCTCGAGTGCGGCACGCGGTTCC 3′)
for the insert.

### c) Purification of Secreted Antibody Fragments.

For purification of and VHH1-secreted antibody fragments, L. paracasei containing the constructs were cultured to an OD₆₀₀ of 0.8. After centrifugation, the pH of the supernatants was adjusted to 7 and filtered through a 0.45 μm filter. The secreted antibody fragments were subsequently purified according to the instructions provided in the RPAS Purification Module (Amersham-Bioscience, Little Chalfont, Buckinghamshire, UK). Dialysis overnight at 8° C. was performed with a Spectra/Por® membrane MWCO 6-8000 (Spectrum Medical Industries, Inc., Los Angeles, Calif.) against 1×PBS. The purified antibody fragments were run on a 15% SDS-poly acrylamide gel to verify the purity of the sample and the concentration of total protein was determined by the BioRad protein assay (BioRad Laboratories, Hercules, Calif.).

### d) Protein Extraction and Determination of Protein Concentration

L. paracasei containing the constructs 2A10-anchor, VHH1-anchor, 2A10-secreted, VHH1-secreted, irrelevant-secreted and irrelevant-anchor were cultured in MRS broth containing 3 μg/ml erythromycin to an OD₆₀₀ of 0.8. The bacteria were lysed in 10 mM Tris-HCl pH 8.0 containing 10 mg/ml lysozyme at 37° C. for one hour and then disrupted by sonication (6×30 s on/off cycles) with 60% duty cycle (Digital Sonifier®, model 250, Branson Ultrasonics coorporation, Danbury, Conn.). Debris was removed by centrifugation. The supernatants were concentrated 50× using ultrafiltration (Amicon, Beverly, Mass.). BioRad protein assay was used to determine the protein concentration as described above.

### e) Enzyme-Linked Immunosorbent Assay and Flow Cytometry

ELISA 96 well plates were coated with rabbit anti-human rotavirus sera (1/1000). 1:100 dilution of Rhesus rotavirus stock (RRV) was used for secondary coating. After blocking, the plates were incubated with the protein extracts or concentrated supernatants. Mouse anti-E-tag antibodies (Amersham Pharmacia Biotech, Bucks, UK) or rabbit anti-llama antibodies, horse raddish (HRP) conjugated goat anti-mouse antibodies or swine anti-rabbit antibodies (DAKO A/S, Glostrup, Denmark) and 3,3′,5,5′-tetramethylbenzidine substrate (TMB) were added and the absorbance was measured at 630 nm using a Vmax Microplate Reader (Molecular Devices, Sunnyvale, Calif.). All antibodies were diluted 1/1000. Purified VHH1-E-tag and monoclonal 2A10 antibodies were used as standard to determine the concentration of antibody fragments produced by the different lactobacilli transformants.

Flow cytometry was carried out according to standard protocols using anti-E-tag antibodies and the samples were analyzed using a FACS Calibur machine (Becton Dickinson, Stockholm, Sweden).

Results are shown in FIG. 4a.

### f) Electron Microscopy SEM TEM.

For Scanning Electron Microscope (SEM) cultures of lactobacilli transformants expressing VHH1 anchored on the surface and the non-transformed L. paracasei were mixed with RRV and after incubation, fixed and added onto a poly-L-lysine coated RC58 coated slide. The slides were analyzed by SEM (JEOL JSM-820, Tokyo, Japan) at 15 kV.

For Transmission Electron Microscope (TEM) RRV were added onto grids, dipped in supernatant (25 times concentrated) from the lactobacilli expressing secreted VHH1 or supernatant from the non-transformed L. paracasei. Subsequently mouse anti-E-tag antibody (1:1000) and 10 nm gold labelled goat anti-mouse IgG antibodies (1:1000) (Amersham Biosciences) were added. The grids were analysed by TEM (Tecnai 10 transmission electron microscope, Fei Company, The Netherlands) at 80 KV.

Results are shown in FIG. 4b and FIG. 11.

### g) Virus Production and Purification.

Rhesus rotavirus was cultured in MA104 cells as previously described. Plaque-purified RRV was used throughout the study. A single virus stock was produced for the entire study by infecting MA104 cells with RRV at a multiplicity of infection (MOI) of 0.1 in serum-free M199 medium (Gibco Laboratories, Grand Island, N.Y.) containing 0.5 μg of trypsin (Sigma Chemical Co., St. Louis, Mo.) per ml. When the cytopathogenic effect reached approximately 75% of the monolayer, cells were freeze-thawed twice and cell lysates were cleared by low-speed centrifugation. The virus suspension was divided into aliquots and stored at −80° C. until use. Determination of virus titers was performed by an immunoperoxidase focus reduction test. A single virus stock was produced for the entire study.

### h) In Vitro Neutralization Assays.

Antibody expressing lactobacilli were further tested for inhibitory effect on rotavirus by a microneutralization assay as described previously (Giammarioli et al 1996 as above). For anchored antibody fragments, the bacteria were serially diluted in MEM media and incubated for 1 h at 37° C. with 200 ffu of trypsin-activated RRV (100 μl). At the end of incubation, bacteria were removed by centrifugation and the supernatant was used for inoculating MA104 cell monolayers grown in 96-well plates. Concentrated culture supernatants from lactobacilli secreting antibody fragments, neat or diluted in MEM, were used for incubation with RRV and inoculation of MA104 cell monolayers. The inoculated plates were incubated at 37° C. for 1 hrs, washed with MEM medium, supplied with fresh MEM medium supplemented with antibiotics (gentamycin and penicillin/streptomycin) and incubated at 37° C. in a CO₂ atmosphere for 18 h. Monolayers were fixed and stained with immunoperoxidase as described (Svensson et al 1991 as above). A reduction in the number of RRV-infected cells greater than 60% with respect to the number in control wells was considered to indicate neutralization. Purified VHH1 fragments produced by lactobacilli were used as a positive control.

Results are shown in FIG. 5.

### i) In Vivo Assays.

All animal experiments were approved by the local ethical committee of the Karolinska Institutet at Huddinge Hospital, Sweden. Pregnant BALB/c female mice were purchased from Møllegard, Denmark. Four-day-old pups were used for the study. Pups were fed 10 μl of different treatments once a day, starting on day −1 until day 3. Lactobacilli were administered once, one day before rotavirus challenge. Infections were made orally on day 0 using 2×10⁷ ffu RRV in 10 μl volume.

Occurrence of diarrhoea was recorded daily until day 4. Pups were euthanized using intra-peritoneal pentobarbital on day 5. Sections of small intestines were stabilized in RNAlater® (QIAGEN) for RNA isolation or fixed in neutral buffered formalin for histopathological analysis or resuspended in sterile PBS for the Lactobacillus survival study. Four independent experiments were conducted with various lactobodies, initially testing the dose response behaviour of the bacteria producing VHH1 anchored VHH1 fragments and subsequently testing other lactobodies at the optimal dose. Control lactobodies expressing irrelevant antibody fragments or nontransformed lactobacilli were included in each experiment. An infection only group was also included in each experiment.

To evaluate the survival of lactobacilli in the intestine of mice, pups were once fed lactobacilli expressing anchored VHH1 on day −1 and half of them were infected with RRV on day 0. Two pups in each group were euthanized and sections of the small intestine were removed on days 1, 3, 7, 14. The presence of transformants was determined by culturing intestinal extracts on Rogosa plates containing erythromycin (3 μg/ml). PCR was used for detection of the VHH1 insert.

Results are shown in FIG. 6.

### j) Analysis of Intestinal Specimens.

Sections of the small intestine were taken on day 4 and perfused with 4% neutral buffered formalin and Hematoxylin and Eosin staining was performed after sectioning according to standard protocols. Individual slides were evaluated blindly for typical signs of rotavirus infection.

Total cellular RNA was isolated from small intestinal tissue and was used for Real Time analysis after digestion of residual genomic DNA using RNase free DNase®. EZ RT-PCR® core reagent kit (PE Applied Biosystems, Foster City, Calif., USA) was used for Real Time PCR. A standard curve was generated using a pet28a (+) vector with the RRV vp7 gene cloned between the NcoI and XhoI restriction sites. Rotavirus vp7 mRNA or viral genomic RNA was amplified at 58° C. (ABI 7000 cycler, Applied Biosystems) in the presence of 600 nM primers, 300 nM probe, 5 mM Mn to generate a 121 bp long amplicon. The sense primer (VP7f: 5′-CCAAGGGAAAAT GTAGCAGTAATTC-3′) (nucleotide (nt) 791-815), the antisense primer (VP7r 5′-TGCCACCATTCTTT CCMTTAA-3′), (nt 891-912), and the probe (5′-6FAM-TMCGGCTGATCCAACCACAGCACC -TAMRA-3′ (nt 843-867) were designed based on the vp7 gene sequence of rhesus rotavirus (accession number AF295303). The lowest level of detection of the PCR is 10 viral RNA copies. The RNA samples from each animal was normalized for the internal housekeeping gene control GAPDH (Overbergh et al, (1999) Cytokine 11: 305-312). Detection of no virus or less than 10 virus genomes was defined as clearance from infection.

Results are shown in FIG. 7.

k) In situ Expression of the VHH1 Fragments on the Lactobacilli Surface in the Feaces.

Feacal samples of three animals from the groups receiving the lactobacilli expressing the VHH1 anchored fragments, non-transformed lactobacilli or a non treated group were collected at the day of termination. The samples were smeared on a Super frost coated glass slide and fixed by methanol:acetone (1:1) for 10 minutes on ice. A mouse anti-E-tag antibody ( 1/200) and thereafter a cy2 labelled donkey anti-mouse antibody ( 1/200) was added to the slides and incubated for 1 hour under humid conditions. The surface expressed VHH1 fragments were detected by fluorescence microscope.

### Statistics

The diarrhoeal illness in pups was assessed on the basis of consistency of feces. Watery diarrhoea was given a score 2 and loose stool was given a score 1, no stool or normal stool was given a score 0. The percentage of diarrhoea score was calculated each day. Total daywise score in a treatment group was compared to untreated group by Fisher's exact test. Severity was defined as the sum of diarrhoea score for each pup during the course of the study and duration was defined as the sum of days with diarrhoea. Both severity and duration were analysed by Kruskal Wallis and Dunn tests.

### Results:

### Discussion of Figures and Tables

TABLE 2
Duration and Severity of diarrhoea in different treatment groups.

| | Duration | | Severity | |
|---|---|---|---|---|
| | (mean ± SE) | P | (mean ± SE) | P |
| VHH1 ank | 1.222 ± 0.163 | Vs | 1.667 ± 0.250 | Vs untreated <0.001 |
| (27) | | untreated <0.01 | | Vs irrelevant |
| | | Vs irrelevant | | <0.01 |
| | | <0.05 | | |
| Untreated (30) | 2.133 ± 0.104 | — | 3.733 ± 0.1656 | — |
| L. paracasei | 1.941 ± 0.200 | — | 2.882 ± 0.352 | — |
| (17) | | | | |
| Irrelevant VHH | 2.118 ± 0.169 | — | 3.353 ± 0.283 | — |
| ank (17) | | | | |
| VHH1 sec (10) | 1.909 ± 0.162 | — | 2.727 ± 0.237 | — |
| 2A10 ank (10) | 2.000 ± 0.258 | — | 2.600 ± 0.3712 | — |
| 2A10 sec (10) | 2.100 ± 0.233 | — | 2.900 ± 0.233 | — |
| Preincubated | 1.200 ± 0.249 | — | 1.700 ± 0.395 | Vs |
| VHH1 ank | | | | untreated <0.01 |
| (10) | | | | |
| Lyophilized | 1.286 ± 0.285 | — | 1.857 ± 0.404 | Vs untreated |
| VHH1 ank (7) | | | | <0.05 |

FIG. 4a shows the surface expression of the 2A10-scFv and VHH1 by lactobacilli was shown by flow cytometry using an anti-E-tag antibody A lower level of detection of the E-tag was observed on lactobacilli producing the 2A10 anchored fragments compared to the VHH1 anchored and irrelevant scFv and VHH control fragments expressing bacteria (data not shown).

The binding activity of the antibody fragments was analyzed by ELISA and electron microscopy. For ELISA, homogenates of 2A10-anchor- and VHH1-anchor-transformed lactobacilli and supernatant from the 2A10-secreted- and VHH1-secreted-transformed lactobacilli were tested using the E-tag for detection. Antibody fragments, VHH1-anchored, VHH1-secreted and 2A10-anchored, expressed from lactobacilli bound to plates coated with rotavirus. A higher level of binding was observed for the llama VHH1 fragments, both anchored and secreted (purified and from the supernatant). The amount of secreted 2A10 was too low to be detected by ELISA. The non-transformed L. paracasei, irrelevant antibody fragments from transformed lactobacilli expressing anchored or secreted scFv and anchored or secreted VHH did not bind to rotavirus (data not shown). The amount of antibody fragments produced by the VHH1-anchor transformants was calculated to be approximately 10⁴ VHH1 fragments/bacteria, and 600 2A10 fragments/bacteria (intracellular and on the surface). The VHH1-secreted transformants produced approximately 1 μg/ml of VHH1 fragments in the supernatant.

FIGS. 4b and 11a show lactobacilli expressing VHH1 antibody fragments on their surface, which were incubated with rotavirus and then analyzed by SEM. The results showed binding of the virus on the bacterial surface (FIG. 4ba and 11b), but not to a non-transformed L. paracasei strain (FIG. 11b). Using TEM (negative staining), binding to the virus by llama antibody fragments from the supernatant of lactobacilli transformed with the VHH1-secreted vector could be demonstrated, whereas the non-transformed L. paracasei strain control supernatant did not bind rotavirus (data not shown).

The effect of Lactobacillus produced antibody fragments in a rotavirus neutralization assay was analysed in FIG. 5. The solid line of this figure represents neutralization level achieved by lactobacilli produced E-tag purified VHH1 antibody (20 μg/ml). Dotted line indicates the neutralization level of 2A10 monoclonal hybridoma supernatant (147 ng/ml). Neutralization achieved by different concentrations of VHH1 anchored lactobacilli (▪), 2A10 anchored lactobacilli (▪) and non-transformed lactobacilli (□).

FIG. 5 shows a significant dose-dependent reduction of the infection in the presence of lactobacilli expressing surface bound VHH1 or in the presence of the supernatant containing the secreted VHH1. A slight neutralizing capacity of the supernatant from non-transformed lactobacilli was also observed. The 2A10-transformed lactobacilli (secreted and anchored) were not protective even though the supernatant of the 2A10 monoclonal hybridoma cells, containing 150 ng/ml of the antibody was 95% protective.

FIG. 6 shows the prevalence of diarrhoea in mice trated with lactobacilli expressing VHH1-anchored fragments. Surface VHH1 expressing lactobacilli significantly reduced the diarrhoea prevalence on day 2 over non transformed lactobacilli (P=0.0172).

FIG. 7 shows that in the untreated group, histology of the duodenum and jejunum sections reveals typical signs of rotavirus infection; swelling of villus tips, vacuolization, constriction of villus bases and unpolarized nuclei within cells (a). The groups receiving L. paracasei (b) or lactobacillus expressing VHH1-anchored (c) and the uninfected (d) shows fairly normalized histology.

FIG. 8 shows that the mean viral load in VHH1 anchored treatment group is at least 200 fold lower than untreated group. A probiotic effect of irrelevant lactobacilli controls was also seen (10 fold reduction in viral load). Clearance from virus was defined as no vp7 detection or detection of less than 10 vp7 RNA molecules. 27% animals were cleared of rotavirus in VHH1 anchored treatment group as compared to 9% in untreated group.

FIG. 9 shows that on day 2 dose 10⁸ CFU and 10⁹ CFU of lactobacilli expressing VHH1-anchored fragments cause significant reduction in diarrhea prevalence compared to untreated group, P<0.0001 and P=0.0024. Number of pups in each group: 7 each in 10⁷ CFU/dose and 10⁸ CFU/dose and 8 in 10⁹ CFU/dose and untreated.

FIG. 10 shows that a group where infections were made with RRV incubated with lactobacilli expressing VHH1-anchored fragments was included. Treatment in this group was continued as usual. Surface VHH1 expressing lactobacilli given in a freeze-dried form significantly reduced the diarrhea prevalence on day 2 compared to the untreated group (P=0.0317). On day 3, there was a significant reduction in diarrhea prevalence in groups receiving preincubated VHH1-anchored expressing lactobacilli (n=7; P=0.0004), lyophilized VHH1-anchored expressing lactobacilli (n=7; P=0.0072), VHH1-anchored expressing lactobacilli (n=10; P=0.0022) in comparison to the untreated group (n=10). Disease severity, in comparison to the untreated group, was also reduced when VHH1 surface expressing Lactobacillus was administered in a freeze-dried form (n=10; P<0.01) or when infections were made with RRV preincubated with a fresh culture of these bacteria for 2 h (P<0.05).

### In Situ Expression of VHH1 Fragments on the Lactobacilli Surface in Faeces (Example 4 (k) Results)

Faecal samples of three animals from the groups receiving lactobacilli expressing the VHH1 anchored fragments, untreated group and negative control mice were collected on day 4, the day of termination, for determination of in situ expression of the VHH1 anchored fragments. Lactobacilli expressing VHH1 could be detected using fluorescent anti-E-tag antibody in the of treated mice. No staining could be observed in the control group (data not shown).

### Survival of Lactobacilli in the Mouse Intestine

Pups were fed lactobacilli expressing the anchored VHH1 against rotavirus once (on day 0) and half of them were subsequently infected with RRV on day 1. Two pups in each group were sacrificed every second day and checked for the presence of Lactobacillus transformants by culturing of the intestinal content. The bacteria could be detected in the duodenum and the ileum 48 h post treatment with no difference between the rotavirus infected and uninfected groups, whereas at 96 h post treatment, no transformants could be detected (data not shown).

### The Efficacy of Lactobacillus Transformants to Reduce Diarrhea in a Rotavirus Infection Model

The therapeutic effect of the transformed lactobacilli was tested in a mouse pup model of rotavirus-induced diarrhea. Pups were orally fed lactobacilli expressing the 2A10-scFv or the VHH1 in secreted or anchored forms during five days (day −1 to 3) and infected with RRV on day 0. Control groups included non-transformed L. paracasei and bacteria expressing an irrelevant anchored VHH antibody fragment. 10⁸ CFU as the optimal dose for diarrhea intervention (FIG. 9). The surface VHH1 expressing bacteria shortened the disease duration (normal duration 1.2 days) by approximately 0.9 days (P<0.01) and by 0.7 days compared to mice treated with non-transformed L. paracasei (P<0.05). The severity of the diarrhea was also reduced from 3.7 to 1.7 in mice treated with surface VHH1 expressing lactobacilli as compared to the disease severity in untreated pups (P<0.001) and by a factor of 1.2 in comparison to pups treated with non-transformed L. paracasei (P<0.01). A minor probiotic effect by the non-transformed lactobacilli was also observed (Table 2). In addition, the diarrhea prevalence was significantly lowered both on days 2 and 3 in mice treated with the Lactobacillus expressing surface VHH1 in comparison to untreated mice (P<0.0001, for both days) or mice treated with non-transformed L. paracasei (P<0.02, for day 2) (FIG. 6a). The constructs expressing the secreted or anchored 2A10 as well as the secreted VHH1 did not induce significantly higher protection than non-transformed lactobacilli (FIG. 6a,b). Disease severity, in comparison to the untreated group, was also reduced when VHH1 surface expressing Lactobacillus was administered in a freeze dried form (P<0.01) or when infections were made with RRV preincubated with a fresh culture of these bacteria for 2 h (P<0.05) (Table 2 and FIG. 5).

Histological examination of proximal small intestine sections on day 4 revealed a marked reduction of pathological changes in rotavirus infected animals treated with surface VHH1 expressing lactobacilli and in some mice, the histology was completely normalized. The histology in the group receiving no lactobacilli revealed typical signs of rotavirus infection with swelling of villus tips, constriction of villus bases, vacuolization and irregularly placed cell nuclei (FIG. 7a,b,c,d). To assess whether there was a reduction in viral replication in the enterocytes, a real time PCR for expression of the rotavirus vp7 gene was developed. The mean viral load in animals receiving lactobacilli expressing surface bound VHH1 antibody fragments was at least 250 fold lower than in untreated mice. Lactobacilli expressing an irrelevant VHH fragment also reduced the viral vp7 load (up to 10 fold). The clearance rate was 27% in the group given lactobacilli expressing surface anchored VHH1 as compared to 9% in the untreated group (FIG. 8).

These experiments show the successful expression of llama derived VHH antibody fragment (VHH1) and scFv (scFv-2A10) against rotavirus both on the surface of Lactobacillus casei 393 pLZ15 and as a secreted protein. Efficacy of these recombinant lactobacilli in the treatment of rotavirus by in vitro neutralization and in an infant mouse infection model has also been demonstrated.

### Example 5

Calcium Alginate Encapsulation of Anti-Rotavirus VHH

A solution of 2% sodium alginate was added dropwise to a solution of 0.1 M calcium chloride, containing 1% of the VHH, resulting in the formation of calcium alginate beads (with a size of about 2 mm). The dispersion was allowed to stand for 30 min for the calcium alginate beads to settle at the bottom of the beaker. The beads were then collected with a sieve and washed once with water

### Example 6

### Compositions and Preparations of Ice Creams Containing Encapsulated Anti-Rotavirus VHH or a Lactobacillus Producing this VHH and Probiotic Bacteria

The following example of an ice cream composition is a food product according to the invention;

| | weight % |
|---|---|
| Sucrose | 13.000 |
| Skimmed Milk Powder | 10.000 |
| Butter fat | 8.000 |
| Maltodextrin 40 | 4.000 |
| Monoglycerol Palmitate (MGP) | 0.300 |
| Locust Bean Gum | 0.144 |
| Carageenan L100 | 0.016 |
| Flavour | 0.012 |

Encapsulated VHH solution at a volume resulting in between 5 and 5000 microgram of VHH per serving.

Probiotic bacteria*¹ at an amount of between 10⁶ and 10¹¹ per 100 g of the ice-cream composition,

Water to 100 *¹ The probiotic bacteria may be any of the types mentioned in the detailed description.
Total soluble solids; 35% by weight
Ice content at −18° C.; 54% by weight

All the ice cream ingredients are mixed together using a high shear mixer for approximately 3 minutes. The water is added at a temperature of 80° C. The temperature of the water ice mix is approximately 55-65° C. after mixing.

The mix is then homogenized (2000 psi) and passed through to a plate heat exchanger for pasteurization at 81° C. for 25 seconds. The mix is then cooled to approximately 4° C. in the plate heat exchanger prior to use.

Alternatively, an anti-rotavirus VHH producing Lactobacillus strain can be added Instead of the encapsulated VHH solution, preferably in a concentration of 10⁹ per serving or higher.

The ice cream pre-mix is then frozen using a Technohoy MF 75 scraped surface heat exchanger, e.g. with no overrun introduced into the ice cream. The ice cream can be extruded at a temperature of from −4.4° C. to −5.4° C. The product can then be hardened in a blast freezer at −35° C., then stored at −25° C.

A water ice solution having the following composition was prepared as follows;

| | % by weight |
|---|---|
| Sucrose | 25 |
| Locust Bean Gum | 0.5 |

Encapsulated VHH solution at a volume resulting in between 5 and 5000 microgram of VHH per serving.

Probiotic bacteria*¹ at an amount of between 10⁶ and 10¹¹ per 100 g of the ice-cream composition,

| | |
|---|---|
| water | to 100 |
| Total soluble solids; | 25.5% by weight |
| Ice content at −18° C.; | 62% by weight |

All the water ice ingredients are mixed together using a high shear mixer for approximately 3 minutes. The water is added at a temperature of 80° C. The temperature of the water ice mix is approximately 55-65° C. after mixing.

The mix is then homogenized (2000 psi) and passed through to a plate heat exchanger for pasteurization at 81° C. for 25 seconds. The mix is then cooled to approximately 40° C. in the plate heat exchanger prior to use.

Instead of the encapsulated VHH solution at this moment also a anti-rotavirus VHH producing Lactobacillus strain can be added preferably in a concentration of 10⁹ per serving or higher.

The water ice solution may be frozen in a Technohoy MF 75 scraped surface heat exchanger with an overrun (volume fraction of air) of 30%. The water ice may be extruded at a temperature of from −3.8° C. to 4.5° C. The product may then be hardened in a blast freezer at −35° C., and stored at −25° C.

### Example 7

### Compositions for Spreads Containing Encapsulated Anti-Rotavirus VHH or a Lactobacillus Producing this VHH and Probiotic Bacteria

Spreads were made according to standard procedure as known in the art, using the compositions as given in Table 3.

TABLE 3
Spread compositions

| | Amount | Amount | Amount | Amount |
|---|---|---|---|---|
| | (wt. %) | (wt. %) | (wt. %) | (wt. %) |
| Component | Example 1 | Example 2 | Example 3 | Example 4 |
| Fat blend | 39.71 | 39.71 | 39.71 | 39.71 |
| Bolec ZT | 0.05 | 0.05 | 0.05 | 0.05 |
| Hymono 8903 | 0.16 | 0.16 | 0.16 | 0.16 |
| β-carotene | 0.08 | 0.08 | 0.08 | 0.08 |
| (1% in Sunflower oil) | | | | |
| Total fat phase | 40.00 | 40.00 | 40.00 | 40.00 |
| Tap water | up to 60 | up to 60 | up to 60 | up to 60 |
| Sour whey powder | 0.27 | 0.27 | 0.27 | 0.27 |
| NaCl | 0.48 | 0.48 | 0.48 | 0.48 |
| K-sorbate | 0.12 | 0.12 | 0.12 | 0.12 |
| Gelatin | 1.10 | 1.10 | 1.10 | 1.10 |
| Citric acid | To pH 4.6 | To pH 5.0 | To pH 4.6 | To pH 5.0 |
| Xanthan gum | 0.10 | 0.10 | 0.10 | 0.10 |
| Calcium salt | 1.74 | 1.74 | 1.27 | 1.27 |
| TCP C13-13 | | | | |
| CaSO4.0.5H2O | | | | |
| PH water phase set | 4.6 | 5.0 | 4.6 | 5.0 |
| Encapsulated VHH | 5-5000 | 5-5000 ug | 5-5000 ug | 5-5000 ug |
| sol. | 10⁶-10¹¹ | 10⁶-10¹¹ | 10⁶-10¹¹ | 10⁶-10¹¹ |
| | per | per | per | per |
| Probiotic bacteria*¹ | 100 g | 100 g | 100 g | 100 g |
| Total water phase | To 100 | To 100 | To 100 | To 100 |
| Total | 100.00 | 100.00 | 100.00 | 100.00 |

Instead of the encapsulated VHH solution after the last heating step also a anti-rotavirus VHH producing Lactobacillus strain can be added aseptically, preferably in a concentration of 10⁹ per serving or higher.

1. A food product or pharmaceutical preparation comprising i) antibodies or antibody fragments which are active in the gut and ii) probiotic micro-organisms independent from the antibodies or antibody fragments.

2. A food product or pharmaceutical preparation according to claim 1 wherein the antibodies or antibody fragments comprise part of a delivery system for delivering the antibodies or antibody fragments to the GIT.

3. A food product or pharmaceutical preparation of according to claim 2 wherein the delivery system comprises encapsulated antibodies or antibody fragments which are released in the gut.

4. A food product or pharmaceutical preparation according to claim 2 or claim 3 wherein the delivery system comprises a micro-organism transformed to be able to produce antibodies or fragments thereof.

5. A food product or pharmaceutical preparation according to claim 4 wherein the antibodies or antibody fragments are expressed and/or secreted in the gut.

6. A food product or pharmaceutical preparation according to claim 5 wherein the micro-organism is a probiotic micro-organism, preferably a lactic acid bacterium, mould or a yeast.

7. A food product or pharmaceutical preparation according to claim 6 wherein the micro-organism is Lactobacillus or Bifidobacterium.

8. A food product or pharmaceutical preparation according to claim 7 wherein the Lactobacillus is Lactobacillus casei.

9. A food product or pharmaceutical preparation according to claim 1 or claim 7 wherein the antibodies are heavy chain immunoglobulins or fragments thereof of the VHH or VNAR type or domain antibodies (dAbs) of the heavy or light chains of immunoglobulins or fragments thereof.

10. A food product or pharmaceutical preparation according to claim 1 or claim 9 wherein the antibodies or antibody fragments are present in an effective amount to treat, reduce or prevent diarrhoea in a subject consuming the food product or pharmaceutical preparation.

11. A food product or pharmaceutical preparation according to claim 10 wherein the probiotic microorganisms (ii) are viable micro-organisms.

12. A food product or pharmaceutical preparation according to claim 1 wherein the probiotic microorganisms (ii) are non-viable micro-organisms.

13. A food product or pharmaceutical preparation according to claim 1 wherein the probiotic microorganisms (ii) comprise probiotic bacterium, probiotic yeasts and/or probiotic moulds.

14. A food product or pharmaceutical preparation according to claim 13 wherein the probiotic bacterium (ii) comprise Lactobacillus and/or Bifido bacterium.

15. A method for making a food product or pharmaceutical preparation according to claim 1 comprising adding the antibodies or antibody fragments and the probiotic microorganisms during the manufacture of the food product or pharmaceutical preparation or an ingredient thereof.

16. (canceled)

17. (canceled)

18. (canceled)

19. (canceled)

20. A method of delivering health benefits to the gut of a subject comprising administering the food product or pharmaceutical preparation according to claim 1 to a subject in need thereof.

21. A dispensing implement for use with a food product comprising probiotic micro-organisms wherein the dispensing implement is coated on at least one surface with antibodies or anti-body fragments which are active in the gut.

22. A dispensing implement according to claim 21, wherein the antibodies or antibody fragments comprise a delivery system for delivering the antibodies or antibody fragments to the GIT.

23. A dispensing implement for use with a food product wherein the dispensing implement is coated on at least one surface with antibodies or anti-body fragments which are active in the gut and probiotic micro-organisms.

24. A dispensing implement according to claim 23, wherein the antibodies or antibody fragments comprise a delivery system for delivering the antibodies or antibody fragments to the GIT.

25. A dispensing implement as claimed in claim 21 wherein the delivery system comprises encapsulated antibodies or antibody fragments.

26. A dispensing implement as claimed in claim 21 wherein the delivery system comprises a micro-organism transformed to be able to produce antibodies or fragments thereof.

27. A dispensing implement as claimed in claim 21 wherein the dispensing implement is a knife, fork, spoon, tube, drinking straw or stick.

28. A food product or pharmaceutical preparation according to claim 9 wherein the antibodies are derived from Camelis.

29. A food product or pharmaceutical preparation according to claim 28 wherein the antibodies are derived from llama heavy chain antibodies or fragments thereof.

30. The method of claim 20 used for the management of enteropathogenic micro-organisms.

31. The method of claim 30 wherein the antibodies or antibody fragments are llama heavy chain antibodies or fragments thereof and the health benefit delivered is an anti-diarrhoeal effect.

32. The method of claim 20 used for the management of rotavirus infection.
